(19) United States
(12) Patent Application Publication
(10) Pub. No.: US 2008/0015225 A1
(43) Pub. Date: Jan. 17, 2008

(54) Dimeric Piperidine Derivatives

(75) Inventors: Miroslav Cik, Boechout (BE); Gaston Stanislas Marcella Diels, Ravels (BE); Guy Rosalia Eugeen Van Lommen, Berlaar (BE)

Correspondence Address: Philip S Johnson;Johnson & Johnson, One Johnson & Johnson Plaza, New Brunswick, NJ, 08933-7003, US

(73) Assignee: JANSSEN PHARMACEUTICA N.V., Beerse (BE)

(21) Appl. No.: 11/632,479

(22) PCT Filed: Jul. 13, 2005
(86) PCT No.: PCT/EP05/53351

§ 371 Date: Jan. 16, 2007

Related U.S. Application Data
(60) Provisional application No. 60/588,711, filed on Jul. 16, 2004.

(30) Foreign Application Priority Data
Jul. 16, 2004 (EP) 04103412.5

(51) Int. Cl. A61K 31/445; A61P 25/00; C07D 401/00

(52) U.S. Cl. 514/316; 546/186; 546/187

(57) ABSTRACT

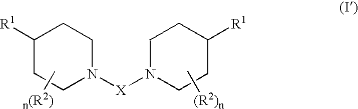

the N-oxide forms, the pharmaceutically acceptable addition salts and the stereochemically isomeric forms thereof, wherein n is 0, 1 or 2;
R² represents hydroxy;
—X— represents C₂₋₄alkynyl, C₁₋₁₂alkyl optionally substituted with hydroxy or X represents a divalent radical of the formula 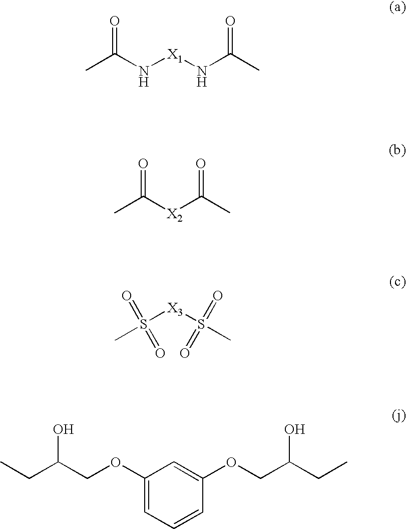
   wherein; —X₁— represents C₁₋₁₂alkyl, phenyl or a divalent radical selected from the group consisting of 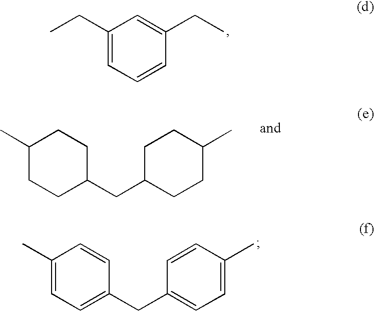
   —X₂— represents C₁₋₁₂alkyl, C₁₋₄alkyloxyC₁₋₄alkyl, phenyl or a divalent radical of formula 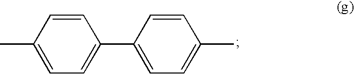
   —X₃— represents phenyl or a divalent radical selected from the group consisting of 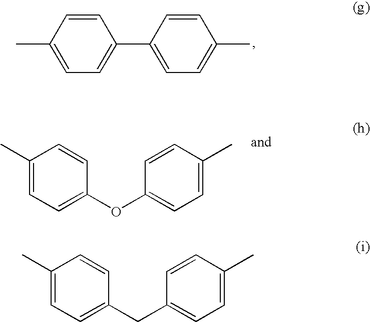
R¹ independently represents hydrogen, C₁₋₄alkyl, C₁₋₄alkyloxy-, Ar¹, Ar²-carbonyl, Het¹-C₁₋₄alkyl, Het², NR³R⁴—C₁₋₄alkyl, Ar³-C₁₋₄alkyloxy- or Het⁴-oxy-;
R³ and R⁴ each independently represents hydrogen, C₁₋₄alkyl, C₁₋₄alkyloxy-, or Het³;
Het¹ represents a heterocycle selected from pyridinyl, indolinyl, indolyl, benzimidazolyl, benzthiazolyl, benzisoxazolyl, thiazolyl, pyridinyl, or thiadiazolyl wherein said Het¹ is optionally substituted with one or where possible two or more substituents selected from the group consisting of hydroxy, halo, C₁₋₄alkyloxycarbonyl-, C₁₋₄alkyl-, C₁₋₄alkyloxy- and C₁₋₄alkyloxy-substituted with halo; in particular Het¹ represents a heterocycle selected from indolyl or pyridinyl;
Het² represents a heterocycle selected from indolyl, benzisoxazolyl or oxodiazolyl wherein said Het² is optionally substituted with one or where possible two or more substituents selected from the group consisting of hydroxy, halo, C₁₋₆alkyl- and C₁₋₄alkyloxy-;
Het³ represents a heterocycle selected from benzimidazolyl, benzisoxazolyl or benzthiazolyl wherein said Het³ is optionally substituted with one or where possible two or more substituents selected from the group consisting of hydroxy, halo, C₁₋₆alkyl- and C₁₋₄alkyloxy-; in particular Het³ represents benzthiazolyl substituted with C₁₋₄alkyloxy-;
Het⁴ represents a heterocycle selected from benzimidazolyl, benzisoxazolyl or benzthiazolyl wherein said Het⁴ is optionally substituted with one or where possible two or more substituents selected from the group consisting of hydroxy, halo, C₁₋₆alkyl- and C₁₋₄alkyloxy-; in particular Het⁴ represents benzthiazolyl;
Ar¹ represents phenyl optionally substituted with halo, C₁₋₄alkyl or C₁₋₄alkyl substituted with one, two or three halo substituents;
Ar² represents phenyl optionally substituted with halo, C₁₋₄alkyl or C₁₋₄alkyl substituted with one, two or three halo substituents; in particular Ar² represents phenyl substituted with halo or trifluromethyl;
Ar³ represents phenyl optionally substituted with halo, C₁₋₄alkyl or C₁₋₄alkyloxy-.

Neurotrophins, such as nerve growth factor (NGF), brain derived growth factor (BDNF), neurotrophic factor 3 (NT3) and neurotrophic factor 4 (NT4) mediate the survival, differentiation, growth and apoptosis of neurons. They bind to two structurally unrelated cell surface receptors, tropomyosin related kinase (Trk) receptors and p75 neurotrophin receptor (p75^{NTR}) (Kaplan D. R. and Miller F. D. (2000) Current Opinion in Neurobiology 10, 381-391). By activating those two type of receptors, neurotrophins mediate both, positive and negative survival signals. NGF binds with high affinity to TrkA, BDNF has high affinity for TrkB, NT-3 binds preferentially to TrkC. Binding of neurotrophins to Trk receptors is necessary for neurotrophic activity. p75^{NTR}, a member of TNF receptor superfamily was first neurotrophin receptor to be described. It binds all neurotrophins with similar affinity. p75^{NTR} was first described as a positive modulator of TrkA activity. Their co-expression lead to an increase of NGF affinity for TrkA receptors, NGF-mediated TrkA activation and ligand specificity. p75^{NTR} can also signal on it own and promote cell death in a variety of cell types. (Coulson E. J., Reid K., and Bartlett P. F. (1999) Molecular Neurobiology 20, 29-44).

Neurotrophins and Possible Therapeutical Relevance

Neurotrophins have a well established role in regulating the survival, differentiation and maintenance of functions of specific and sometimes overlapping neuronal populations. Besides these roles of neurotrophins during embryonic development and adulthood, there is increasing evidence that neurotrophins are involved in processes of neuronal plasticity. These studies suggest several potential therapeutic application. It has been shown that neurotrophins can protect and rescue certain neuronal populations in in vitro and in vivo models of various neurodegenerative diseases such as Alzheimer's disease, Parkinson's disease, Amyotrophic lateral sclerosis (ALS), stroke and peripheral neuropathies (Chao M. V. (2003) Nature Reviews Neuroscience 4, 299-309; Dawbarn D. and Allen S. J. (2003) Neuropathology & Applied Neurobiology 29, 211-230).

In addition, accumulating evidence in last few years shows that p75^{NTR} plays a key role in neuronal death that occurs in some of the major disorders of the CNS such as stroke, Alzheimer's, ALS, epilepsy, Spinal Cord Injury (SCI), Multiple Sclerosis (MS), Motor Neuron Disease (MND) and other neurodegenerative diseases (Park et al. (2000) Journal of Neuroscience 20, 9096-9103; Oh et al. (2000) Brain Research 853, 174-185; Lowry et al. (2001) Journal of Neuroscience Research 64, 11-17; Sedel et al. (1999) European Journal of Neuroscience 11, 3904-3912; Dowling et al. (1999) Neurology 53, 1676-1682) and only recently, NGF was found to play an important role in pain, in particular in post-operative pain after surgery (Zahn et al. 2004, The Journal of Pain 5(3); 157-163). For these reasons small molecules that enhance the activity of neurotrophins, or that have similar effects as neurotrophins, are of great interest (Massa et al. (2002) Journal of Molecular Neuroscience 19,107-11 1; Saragovi and Burgess (1999) Expert Opinion on Therapeutic Patents 9, 737-751).

Experimental Evidence

Peripheral neurons derived from chick embryo dorsal root ganglia (DRG) are extensively used for in vitro characterizations of neurotrophic factors and other molecules with neurotrophic activities. The survival of chick DRG neurons can be supported by different neurotrophic factors, such as nerve growth factor (NGF) (Levi-Montalcini R. and Angeletti P. U. (1968) Physiological Reviews 48, 534-569) brain derived neurotrophic factor (Barde Y. A. et al. (1982) EMBO Journal 1, 549-553) and ciliary neurotrophic factor (CNTF) (Barbin G. et al. (1984) Journal of Neurochemistry 43, 1468-1478). Small molecules with the neurotrophic activity, such as K-252a and CEP-1347 also support the survival of DRG neurons (Borasio G. D. (1990) Neuroscience Letters 108, 207-212; Borasio G. D. et al. (1998) Neuroreport 9, 1435-1439). The primary culture of dissociated DRG neurons from chicken embryo at embryonic day 8-10 has been used successfully in a number of laboratories as a bioassay for neurotrophins. The assay determines the survival effect of compounds on DRG neurons and is based on a fluorimetric Calcein-AM measurement (He W. et al. (2002) Bioorganic & Medicinal Chemistry 10, 3245-3255). This assay, which addresses the functional response of neurons as a quantitative measure of survival, may have the advantage of few false positive.

HTS campaign using a primary culture of chicken DRG neurons, resulted in the identification of compounds with neurotrophic activity (neuronal survival). The most potent compounds identified belong to a series of “symmetrical compounds”.

This invention concerns compounds of formula (I)

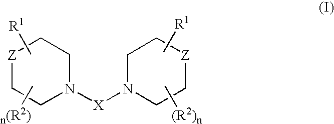

the N-oxide forms, the pharmaceutically acceptable addition salts and the stereochemically isomeric forms thereof, wherein
n is 0, 1 or 2; or
Z represents CH or CH₂;
—X— represents C₂₋₄alkynyl, C₂₋₄alkenyl, C₁₋₁₂alkyl optionally substituted with hydroxy or X represents a divalent radical of the formula 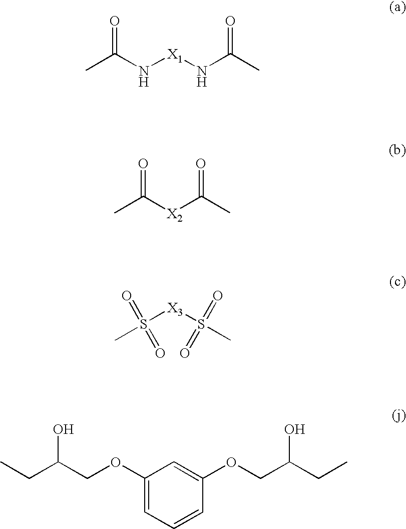
   wherein; —X₁— represents C₁₋₁₂alkyl, phenyl or a divalent radical selected from the group consisting of 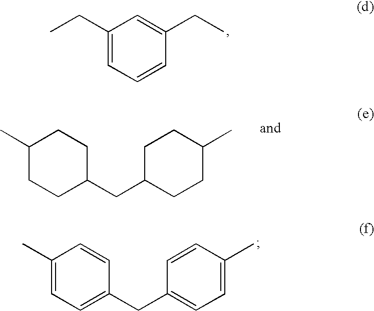
   —X₂— represents C₁₋₁₂alkyl, C₁₋₄alkyloxyC₁₋₄alkyl, phenyl or a divalent radical of formula 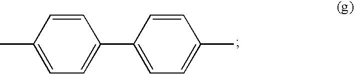
   —X₃— represents phenyl or a divalent radical selected from the group consisting of 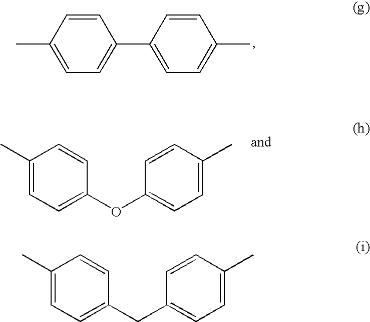
R¹ represents Ar¹, Ar²-carbonyl, Het², Ar³—C₁₋₄alkyloxy-, Ar⁴-oxy-, Het⁴-oxy-, or C₁₋₄alkyl substituted with one and where possible two or three substituents independently selected from NR³R⁴—, Het¹ or Ar⁶; or
R² represents hydroxy, benzyl, or C₁₋₄alkyloxy-;
R³ and R⁴ each independently represents hydrogen, C₁₋₄alkyl, C₁₋₄alkyloxy-, or Het³;
Het¹ represents a heterocycle selected from pyridinyl, pyrimidinyl, indolinyl, indolyl, benzimidazolyl, benzthiazolyl, benzisothiazolyl, benzisoxazolyl, thiazolyl, isothiazolyl or thiadiazolyl wherein said Het¹ is optionally substituted with one or where possible two or more substituents selected from the group consisting of hydroxy, halo, C₁₋₄alkyloxycarbonyl-, C₁₋₄alkyl-, C₁₋₄alkyloxy- and C₁₋₄alkyloxy-substituted with halo; in particular Het¹ represents a heterocycle selected from indolyl or pyridinyl;
Het² represents a heterocycle selected from indolyl, indolinyl, pyridinyl, pyrimidinyl, benzimidazolyl, benzoxazolyl, benzisoxazolyl, quinolinyl, quinazolinyl, quinoxalinyl, or oxodiazolyl wherein said Het² is optionally substituted with one or where possible two or more substituents selected from the group consisting of hydroxy, carbonyl, Ar⁵, halo, C₁₋₆alkyl- and C₁₋₄alkyloxy-;
Het³ represents a heterocycle selected from benzimnidazolyl, benzoxazolyl, benzisoxazolyl, benzisothiazolyl or benzthiazolyl wherein said Het³ is optionally substituted with one or where possible two or more substituents selected from the group consisting of hydroxy, halo, C₁₋₆alkyl- and C₁₋₄alkyloxy-; in particular Het³ represents benzthiazolyl substituted with C₁₋₄alkyloxy-;
Het⁴ represents a heterocycle selected from pyrimidinyl, pyridinyl, indolinyl, indolyl, benzimidazolyl, benzoxazolyl, benzisoxazolyl, benzisothiazolyl or benzthiazolyl wherein said Het⁴ is optionally substituted with one or where possible two or more substituents selected from the group consisting of hydroxy, amino, mono or di-(C₁₋₄alkyl)amino, halo, C₁₋₆alkyl- and C₁₋₄alkyloxy-; in particular Het⁴ represents benzthiazolyl;
Ar¹ and Ar² each independently represent phenyl optionally substituted with halo, C₁₋₄alkyl-, C₁₋₄alkyloxy- or C₁₋₄alkyl substitutedtwith one, two or three halo substituents; in particular Ar² or Ar¹ represents phenyl substituted with halo or trifluromethyl;
Ar³ and Ar⁴ each independently represent phenyl optionally substituted with halo, C₁₋₄alkyl-, C₁₋₄alkyloxy- or C₁₋₄alkyl substituted with one, two or three halo substituents; in particular Ar³ or Ar⁴ represents phenyl substituted with halo or trifluromethyl;
Ar⁵ represents phenyl optionally substituted with halo, C₁₋₆alkyl, C₁₋₄alkyloxy-, or C₃₋₆cycloalkyl-oxy-;
Ar⁶ represents phenyl optionally substituted with halo, C₁₋₆alkyl, C₁₋₄alkyloxy-, or C₃₋₆cycloalkyl-oxy-; provided however that;
for those compounds of formula (I) wherein —X— represents C₁₋₁₂alkyl optionally substituted with hydroxyl and R¹ represents Ar¹, for said compounds n represents 1 or 2; and
for those compounds of formula (I) wherein —X₂— represents phenyl, for said compounds R¹ represents Ar¹, Ar²-carbonyl, Ar³-C₁₋₄alkyloxy-, Ar⁴-oxy-, Het⁴-oxy-, or C₁₋₄alkyl substituted with one and where possible two or three substituents independently selected from NR³R⁴—, Het¹ or Ar⁶.

Dimeric piperidine derivatives have been described before as being useful for the treatment of HCV (WO 97/36554) or as sigma receptor ligands in the treatment of psychosis and movement disorders (WO 93/25527). A possible neurotrophic effect of dimeric piperidine derivatives has never been proposed nor suggested. Surprisingly, the dimeric piperidine derivatives of the present invention, i.e. the compounds of formula (I) and (I′) where found to have a neurotrophic activity. It is accordingly an object of the present invention to provide the use of the compounds of formula (I) or (I′) in the manufacture of a medicament for the treatment or prevention of neurodegenerative disorders.

As used herein before, the terms;

oxo or carbonyl refers to (═O) that forms a carbonyl moiety with the carbon atom to which it is attached;

halo is generic to fluoro, chloro, bromo and iodo;

C₁₋₄alkyl defines straight and branched chain saturated hydrocarbon radicals having from 1 to 4 carbon atoms such as, for example, methyl, ethyl, propyl, butyl, 1-methylethyl, 2-methylpropyl, 2,2-dimethylethyl and the like;

C₁₋₆alkyl is meant to include C₁₋₄alkyl and the higher homologues thereof having 6 carbon atoms such as, for example hexyl, 1,2-dimethylbutyl, 2-methylpentyl and the like;

C₁₋₄alkyloxy defines straight or branched saturated hydrocarbon radicals having from 1 to 4 carbon atoms and 1 oxygen atom such as methoxy, ethoxy, propyloxy, butyloxy, 1-methylethyloxy, 2-methylpropyloxy and the like.

The heterocycles as mentioned in the above definitions and hereinafter, are meant to include all possible isomeric forms thereof, for instance triazolyl also includes 1,2,4-triazolyl and 1,3,4-triazolyl; oxadiazolyl includes 1,2,3-oxadiazolyl, 1,2,4-oxadiazolyl, 1,2,5-oxadiazolyl and 1,3,4-oxadiazolyl; thiadiazolyl includes 1,2,3-thiadiazolyl, 1,2,4-thiadiazolyl, 1,2,5-thiadiazolyl and 1,3,4-thiadiazolyl.

Further, the heterocycles as mentioned in the above definitions and hereinafter may be attached to the remainder of the molecule of formula (I) through any ring carbon or heteroatom as appropriate. Thus, for example, when the heterocycle is imidazolyl, it may be a 1-imidazolyl, 2-imidazolyl, 4-imidazolyl and 5-imidazolyl; when it is thiazolyl, it may be 2-thiazolyl, 4-thiazolyl and 5-thiazolyl; when it is benzothiazolyl, it may be 2-benzothiazolyl, 4-benzothiazolyl, 5-benzothiazolyl, 6-benzothiazolyl and 7-benzothiazolyl.

The pharmaceutically acceptable addition salts as mentioned hereinabove are meant to comprise the therapeutically active non-toxic acid addition salt forms, which the compounds of formula (I), are able to form. The latter can conveniently be obtained by treating the base form with such appropriate acid. Appropriate acids comprise, for example, inorganic acids such as hydrohalic acids, e.g. hydrochloric or hydrobromic acid; sulfuric; nitric; phosphoric and the like acids; or organic acids such as, for example, acetic, propanoic, hydroxyacetic, lactic, pyruvic, oxalic, malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

The pharmaceutically acceptable addition salts as mentioned hereinabove are meant to comprise the therapeutically active non-toxic base addition salt forms which the compounds of formula (I), are able to form. Examples of such base addition salt forms are, for example, the sodium, potassium, calcium salts, and also the salts with pharmaceutically acceptable amines such as, for example, ammonia, alkylamines, benzathine, N-methyl-D-glucamine, hydrabamine, amino acids, e.g. arginine, lysine.

Conversely said salt forms can be converted by treatment with an appropriate base or acid into the free acid or base form.

The term addition salt as used hereinabove also comprises the solvates which the compounds of formula (I), as well as the salts thereof, are able to form. Such solvates are for example hydrates, alcoholates and the like.

The term stereochemically isomeric forms as used hereinbefore defines the possible different isomeric as well as conformational forms which the compounds of formula (I), may possess. Unless otherwise mentioned or indicated, the chemical designation of compounds denotes the mixture of all possible stereochemically and conformationally isomeric forms, said mixtures containing all diastereomers, enantiomers and/or conformers of the basic molecular structure. All stereochemically isomeric forms of the compounds of formula (I), both in pure form or in admixture with each other are intended to be embraced within the scope of the present invention.

The N-oxide forms of the compounds of formula (I), are meant to comprise those compounds of formula (I) wherein one or several nitrogen atoms are oxidized to the so-called N-oxide.

A first group of compounds consist of those compounds of formula (I) wherein;
n is 0, 1 or 2;
Z represents —CH— or —CH₂—;
—X— represents C₂₋₄alkynyl, C₁₋₁₂alkyl optionally substituted with hydroxy or X represents a divalent radical of the formula 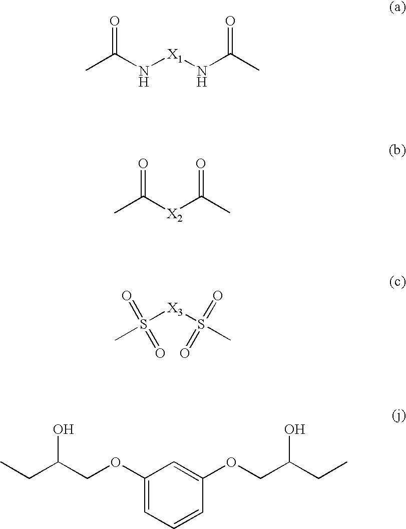
   wherein; —X₁— represents C₁₋₁₂alkyl, phenyl or a divalent radical selected from the group consisting of 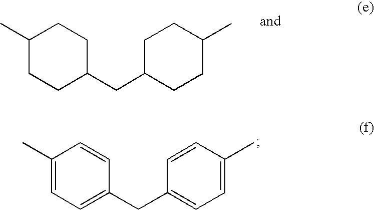
   —X₂— represents C₁₋₁₂alkyl, phenyl or a divalent radical of formula 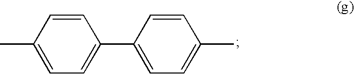
   —X₃— represents phenyl or a divalent radical selected from the group consisting of 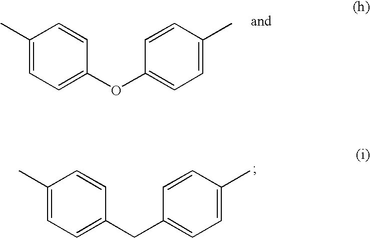
R¹ represents Ar¹, Ar²-carbonyl, Het², Ar³—C₁₋₄alkyloxy-, Het⁴-oxy- or C₁₋₄alkyl substituted with one or where possible two or three substituents independently selected from NR³R⁴ or Het¹;
R² represents hydroxyl;
R³ and R⁴ each independently represent hydrogen or Het³;

Het¹ represents a heterocycle selected from indolinyl, indolyl, pyridinyl, benzthiazolyl or benzisothiazolyl wherein said Het¹ is optionally substituted with one or where possible two or more substituents selected from halo, hydroxyl or C₁₋₄alkyloxy;
Het² represents a heterocycle selected from indolyl, indolinyl, benzoxazolyl, benzisoxazolyl or oxodiazolyl wherein said Het² is optionally substituted with one or where possible two or more substituents selected from halo, hydroxyl, Ar⁵ or C₁₋₆alkyl;
Het³ represents a heterocycle selected from benzthiazolyl or benzisothiazolyl, wherein said Het³ is optionally substituted with one or where possible two or more substituents selected from halo, hydroxyl or C₁₋₄alkyloxy;
Het⁴ represents a heterocycle selected from benzthiazolyl or benzisothiazolyl, wherein said Het³ is optionally substituted with one or where possible two or more substituents selected from halo, hydroxyl or C₁₋₄allyloxy;
Ar¹ and Ar² each independently represent phenyl optionally substituted with one, two or more substituents selected from halo or C₁₋₄alkyl substituted with one, two or three halo substituents;
Ar³ and Ar⁴ each independently represent phenyl optionally substituted with one, two or more substituents selected from halo or C₁₋₄alkyl substituted with one, two or three halo substituents; and
Ar⁵ represents phenyl optionally substituted with C₁₋₄alkyloxy-, or C₃₋₆cycloalkyloxy-; provided however that;
for those compounds of formula (I) wherein —X— represents C₁₋₁₂alkyl optionally substituted with hydroxyl and R¹ represents Ar¹, for said compounds n represents 1 or 2; and
for those compounds of formula (I) wherein —X₂— represents phenyl, for said compounds R¹ represents Ar¹, Ar²-carbonyl, Ar³—C₁₋₄alkyloxy-, Ar⁴-oxy-, Het⁴-oxy-, or C₁₋₄alkyl substituted with one and where possible two or three substituents independently selected from NR³R⁴—, Het¹ or Ar⁶.

A second group of compounds consist of those compounds of formula (I) wherein;
n is 0, 1 or 2; Z represents CH or CH₂;
—X— represents C₂₋₄alkynyl, C₁₋₁₂alkyl optionally substituted with hydroxy or X represents a divalent radical of the formula 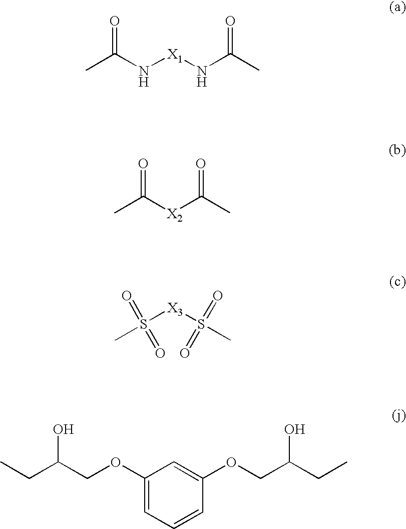
   wherein; —X₁— represents C₁₋₁₂alkyl, phenyl or a divalent radical selected from the group consisting of 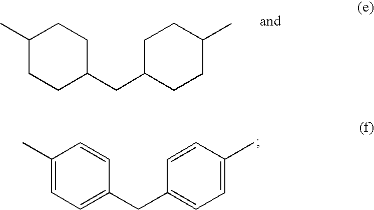
   —X₂— represents C₁₋₁₂alkyl, phenyl or a divalent radical of formula 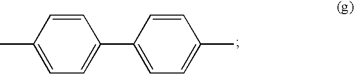
   —X₃— represents phenyl or a divalent radical selected from the group consisting of 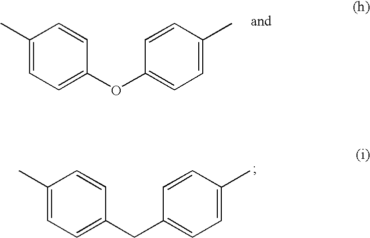
R¹ represents Ar¹, Ar²-carbonyl, Het², Ar³-C₁₋₄alkyloxy-, Het⁴-oxy- or C₁₋₄alkyl substituted with one or where possible two or three substituents independently selected from NR³R⁴ or Het¹;
R² represents hydroxyl;
R³ and R⁴ each independently represent hydrogen or Het³;
Het¹ represents a heterocycle selected from indolyl or benzthiazolyl;
Het² represents a heterocycle selected from indolyl, pyridinyl, benzisoxazolyl or oxodiazolyl wherein said Het² is optionally substituted with one or where possible two or more substituents selected from halo, Ar⁵ or C₁₋₆alkyl;
Het³ represents benzthiazolyl wherein said Het³ is optionally substituted with one or where possible two or more substituents selected from halo or C₁₋₄alkyloxy; in particular Het³ represents benzthiazolyl substituted with one or more C₁₋₄alkyloxy substituents;
Het⁴ represents benzthiazolyl;
Ar¹ and Ar² each independently represent phenyl optionally substituted with one, two or more substituents selected from halo or C₁₋₄alkyl substituted with one, two or three halo substituents;
Ar³ and Ar⁴ each independently represent phenyl optionally substituted with one, two or more C₁₋₄alkyl substituents, said C₁₋₄alkyl substituted with one, two or three halo substituents; and
Ar⁵ represents phenyl optionally substituted with C₁₋₄alkyloxy-, or C₃₋₆cycloalkyl-oxy-; provided however that;
for those compounds of formula (I) wherein —X— represents C₁₋₁₂alkyl optionally substituted with hydroxyl and R¹ represents Ar¹, for said compounds n represents 1 or 2; and
for those compounds of formula (I) wherein —X₂— represents phenyl, for said compounds R¹ represents Ar¹, Ar²-carbonyl, Ar³—C₁₋₄alkyloxy-, Ar⁴-oxy-, Het⁴-oxy-, or C₁₋₄alkyl substituted with one and where possible two or three substituents independently selected from NR³R⁴—, Het¹ or Ar⁶.

A second group of compounds consist of those compounds of formula (I′) wherein;

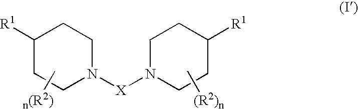

the N-oxide forms, the pharmaceutically acceptable addition salts and the stereochemically isomeric forms thereof, wherein
n is 0, 1 or 2;
R² represents hydroxy;
—X— represents C₂₋₄alkynyl, C₁₋₁₂alkyl optionally substituted with hydroxy or X represents a divalent radical of the formula 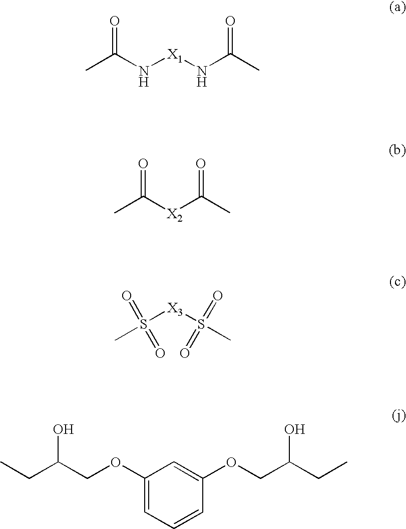
   wherein; —X₁— represents C₁₋₁₂alkyl, phenyl or a divalent radical selected from the group consisting of 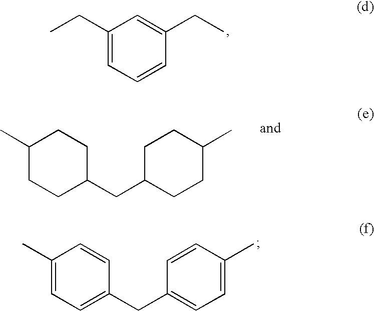
   —X₂— represents C₁₋₁₂alkyl, C₁₋₄alkyloxyC₁₋₄alkyl, phenyl or a divalent radical of formula 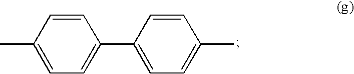
   —X₃— represents phenyl or a divalent radical selected from the group consisting of 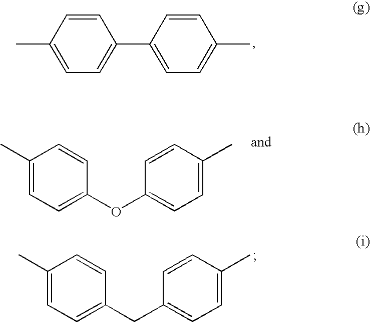
R¹ independently represents hydrogen, C₁₋₄alkyl, C₁₋₄alkyloxy-, Ar¹, Ar²-carbonyl, Het¹-C₁₋₄alkyl, Het², NR³R⁴—C₁₋₄alkyl, Ar³—C₁₋₄alkyloxy- or Het⁴-oxy-;
R³ and R⁴ each independently represents hydrogen, C₁₋₄alkyl, C₁₋₄alkyloxy-, or Het³;
Het¹ represents a heterocycle selected from pyridinyl, indolinyl, indolyl, benzimidazolyl, benzthiazolyl, benzisoxazolyl, thiazolyl, pyridinyl, or thiadiazolyl wherein said Het¹ is optionally substituted with one or where possible two or more substituents selected from the group consisting of hydroxy, halo, C₁₋₄alkyloxycarbonyl-, C₁₋₄alkyl-, C₁₋₄alkyloxy- and C₁₋₄alkyloxy-substituted with halo; in particular Het¹ represents a heterocycle selected from indolyl or pyridinyl;
Het² represents a heterocycle selected from indolyl, pyridinyl, benzisoxazolyl or oxodiazolyl wherein said Het² is optionally substituted with one or where possible two or more substituents selected from the group consisting of hydroxy, halo, C₁₋₆alkyl- and C₁₋₄alkyloxy-;
Het³ represents a heterocycle selected from benzimidazolyl, benzisoxazolyl or benzthiazolyl wherein said Het³ is optionally substituted with one or where possible two or more substituents selected from the group consisting of hydroxy, halo, C₁₋₆alkyl- and C₁₋₄alkyloxy-; in particular Het³ represents benzthiazolyl substituted with C₁₋₄alkyloxy-;
Het⁴ represents a heterocycle selected from benzimidazolyl, benzisoxazolyl or benzthiazolyl wherein said Het⁴ is optionally substituted with one or where possible two or more substituents selected from the group consisting of hydroxy, halo, C₁₋₆alkyl- and C₁₋₄alkyloxy-; in particular Het⁴ represents benzthiazolyl;
Ar¹ represents phenyl optionally substituted with halo, C₁₋₄alkyl or C₁₋₄alkyl substituted with one, two or three halo substituents;
Ar² represents phenyl optionally substituted with halo, C₁₋₄alkyl or C₁₋₄alkyl substituted with one, two or three halo substituents; in particular Ar² represents phenyl substituted with halo or trifluromethyl; and
Ar³ represents phenyl optionally substituted with halo, C₁₋₄alkyl or C₁₋₄alkyloxy-; provided however that;
for those compounds of formula (I′) wherein —X— represents C₁₋₁₂alkyl optionally substituted with hydroxyl and R¹ represents Ar¹, for said compounds n represents 1 or 2; and
for those compounds of formula (I′) wherein —X₂— represents phenyl, for said compounds R¹ represents Ar¹, Ar²-carbonyl, Ar³—C₁₋₄alkyloxy-, Het⁴-oxy-, or C₁₋₄alkyl substituted with one and where possible two or three substituents independently selected from NR³R⁴— or Het¹.

This invention concerns compounds of formula (I)

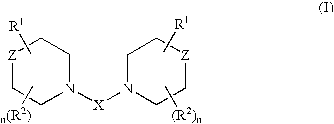

the N-oxide forms, the pharmaceutically acceptable addition salts and the stereochemically isomeric forms thereof, wherein
n is 0, 1 or 2;
Z represents C, N or O; in particular Z represents CH or CH₂;
—X— represents C₂₋₄alkynyl, C₂₋₄alkenyl, C₁₋₁₂alkyl optionally substituted with hydroxy or X represents a divalent radical of the formula 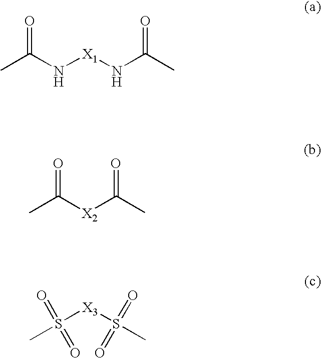
   wherein; —X₁— represents C₁₋₁₂alkyl, phenyl or a divalent radical selected from the group consisting of 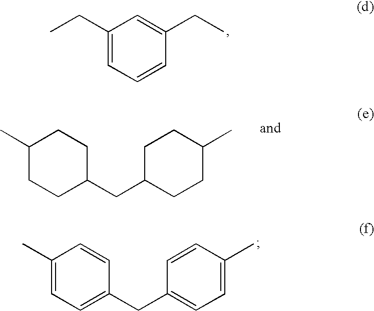
   —X₂— represents C₁₋₁₂alkyl, C₁₋₄alkyloxyC₁₋₄alkyl, phenyl or a divalent radical of formula 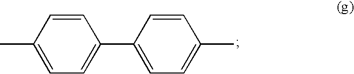
   —X₃— represents phenyl or a divalent radical selected from the group consisting of 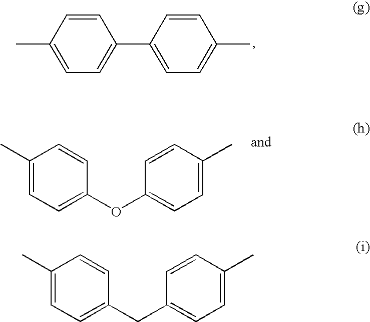
R¹ independently represents hydrogen, C₁₋₄alkyl, Ar¹, C₁₋₄alkyloxy-, Ar²-carbonyl, Het², Ar³—C₁₋₄alkyloxy-, Ar⁴-oxy-, Het⁴-oxy-, or C₁₋₄alkyl substituted with one and where possible two or three substituents independently selected from NR³R⁴—, Het¹ or Ar⁶;
R² represents hydroxy, benzyl, or C₁₋₄alkyloxy-;
R³ and R⁴ each independently represents hydrogen, C₁₋₄alkyl, C₁₋₄alkyloxy-, or Het³;
Het¹ represents a heterocycle selected from pyridinyl, indolinyl, indolyl, benzimidazolyl, benzthiazolyl, benzisoxazolyl, thiazolyl, or thiadiazolyl wherein said Het¹ is optionally substituted with one or where possible two or more substituents selected from the group consisting of hydroxy, halo, C₁₋₄alkyloxycarbonyl-, C₁₋₄alkyl-, C₁₋₄alkyloxy- and C₁₋₄alkyloxy-substituted with halo; in particular Het¹ represents a heterocycle selected from indolyl or pyridinyl;
Het² represents a heterocycle selected from indolyl, indolinyl, imidazolidinyl, benzimidazolyl, benzoxazolyl, benzisoxazolyl, quinolinyl, quinazolinyl, quinoxalinyl, or oxodiazolyl wherein said Het² is optionally substituted with one or where possible two or more substituents selected from the group consisting of hydroxy, carbonyl, Ar⁵, halo, C₁₋₆alkyl- and C₁₋₄alkyloxy-;
Het³ represents a heterocycle selected from benzimidazolyl, benzisoxazolyl or benzthiazolyl wherein said Het³ is optionally substituted with one or where possible two or more substituents selected from the group consisting of hydroxy, halo, C₁₋₆alkyl- and C₁₋₄alkyloxy-; in particular Het³ represents benzthiazolyl substituted with C₁₋₄alkyloxy-;
Het⁴ represents a heterocycle selected from pyrimidinyl, pyridinyl, indolinyl, indolyl, benzimidazolyl, benzisoxazolyl or benzthiazolyl wherein said Het⁴ is optionally substituted with one or where possible two or more substituents selected from the group consisting of hydroxy, amino, mon or di-(C₁₋₄alkyl)amino, halo, C₁₋₆alkyl- and C₁₋₄alkyloxy-; in particular Het⁴ represents benzthiazolyl;
Ar¹ and Ar² each independently represent halo, C₁₋₄alkyl-, C₁₋₄alkyloxy- or C₁₋₄alkyl substituted with one, two or three halo substituents; in particular Ar² represents phenyl substituted with halo or trifluromethyl;
Ar⁵ represents phenyl optionally substituted with halo, C₁₋₆alkyl, C₁₋₄alkyloxy-, or C₃₋₆cycloalkyl-oxy-;
Ar⁶ represents phenyl optionally substituted with halo, C₁₋₆alkyl, C₁₋₄alkyloxy-, or C₃₋₆cycloalkyl-oxy-;

A first group of compounds consist of those compounds of formula (I) wherein one or more of the following restrictions apply;
n is 0, 1 or 2; in a further embodiment
—X— represents C₂₋₄alkynyl, C₁₋₁₂alkyl optionally substituted with hydroxy or X represents a divalent radical of the formula 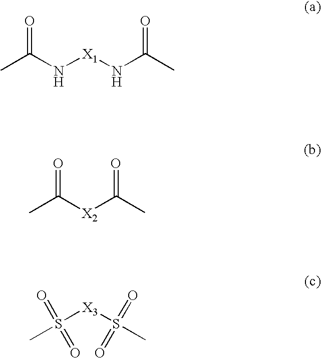
   wherein; —X₁— represents C₁₋₁₂alkyl, phenyl or a divalent radical selected from the group consisting of 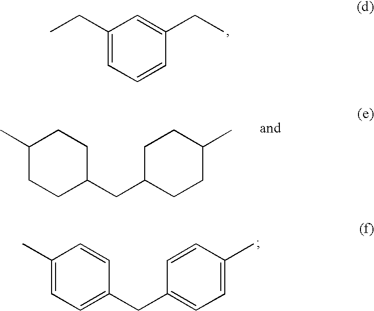
   —X₂— represents C₁₋₁₂alkyl, C₁₋₄alkyloxyC₁₋₄alkyl, phenyl or a divalent radical of formula 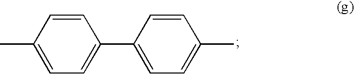
   —X₃— represents phenyl or a divalent radical selected from the group consisting of 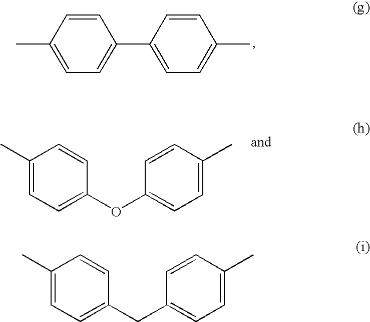
Z represents C or N, in particular CH, CH₂, N or NH;
R¹ independently represents hydrogen, C₁₋₄alkyl, C₁₋₄alkyloxy-, Ar¹, Ar²-carbonyl, Het¹-C₁₋₄alkyl, Het², NR³R⁴—C₁₋₄alkyl, Ar³—C₁₋₄alkyloxy- or Het⁴-oxy-;
R³ and R⁴ each independently represent hydrogen, C₁₋₄alkyl, C₁₋₄alkyloxy-, or Het³;
Het¹ represents a heterocycle selected from pyridinyl, indolinyl, indolyl, benzimidazolyl, benzthiazolyl, benzisoxazolyl, thiazolyl, or thiadiazolyl wherein said Het¹ is optionally substituted with one or where possible two or more substituents selected from the group consisting of hydroxy, halo, C₁₋₄alkyloxycarbonyl-, C₁₋₄alkyl-, C₁₋₄alkyloxy- and C₁₋₄alkyloxy-substituted with halo;
Het² represents a heterocycle selected from indolyl, indolinyl, benzimidazolyl, benzisoxazolyl or oxodiazolyl wherein said Het² is optionally substituted with one or where possible two or three substituents selected from the group consisting of hydroxy, halo, C₁₋₆alkyl, C₁₋₄alkyloxy-, carbonyl and Ar⁵; in particular Het² represents a heterocycle selected from indolyl, benzisoxazolyl or oxodiazolyl wherein said Het² is optionally substituted with one or where possible two or more substituents selected from the group consisting of hydroxy, halo, C₁₋₆alkyl- and C₁₋₄alkyloxy-;
Het³ represents a heterocycle selected from benzimidazolyl, benzisoxazolyl or benzthiazolyl wherein said Het³ is optionally substituted with one or where possible two or more substituents selected from the group consisting of hydroxy, halo, C₁₋₆alkyl- and C₁₋₄alkyloxy-;
Het⁴ represents a heterocycle selected from pyridinyl, indolinyl, indolyl, benzimidazolyl, benzisoxazolyl or benzthiazolyl wherein said Het⁴ is optionally substituted with one or where possible two or more substituents selected from the group consisting of hydroxy, halo, C₁₋₆alkyl- and C₁₋₄alkyloxy-; in particular Het⁴ represents a heterocycle selected from benzimidazolyl, benzisoxazolyl or benzthiazolyl wherein said Het⁴ is optionally substituted with one or where possible two or more substituents selected from the group consisting of hydroxy, halo, C₁₋₆alkyl- and C₁₋₄alkyloxy-;
Ar¹ represents phenyl optionally substituted with halo, C₁₋₄alkyl or C₁₋₄alkyl substituted with one, two or three halo substituents;
Ar² represents phenyl optionally substituted with halo, C₁₋₄alkyl or C₁₋₄alkyl substituted with one, two or three halo substituents;
Ar³ represents phenyl optionally substituted with halo, C₁₋₄alkyl, C₁₋₄alkyloxy- or C₁₋₄alkyl substituted with one, two or three halo substituents;
Ar⁵ represents phenyl optionally substituted with C₁₋₄alkyloxy- or C₃₋₆cycloalkyloxy-.

Another interesting group of compounds are those compounds of formula (I) wherein one or more of the following restrictions apply;
n is 0 or 1;
R² represents hydroxy;
Z represents C or N, preferably CH or CH₂;
R¹ represents Ar¹, Ar²-carbonyl, Het², Ar³—C₁₋₄alkyloxy-, Het⁴-oxy or Het¹-C₁₋₄alkyl;
Het¹ represents a heterocycle selected from pyridinyl, indolinyl, indolyl, benzthiazolyl or benzisoxazolyl wherein said Het¹ is optionally substituted with one or where possible two or more substituents halo and C₁₋₄alkyloxy;
Het² represents a heterocycle selected from indolyl, indolinyl, benzimidazolyl, benzisoxazolyl or oxodiazolyl wherein said Het² is optionally substituted with one or where possible two or more substituents selected from the group consisting of hydroxy, carbonyl, Ar⁵ and halo;
Het⁴ represents benzthiazolyl;
Ar¹ represents phenyl optionally substituted with C₁₋₄alkyl substituted with one, two or three halo substituents;
Ar² represents phenyl optionally substituted with halo or C₁₋₄alkyl substituted with one, two or three halo substituents;
Ar³ represents phenyl optionally substituted with C₁₋₄alkyl substituted with one, two or three halo substituents; or
Ar⁵ represents phenyl optionally substituted with C₁₋₄alkyloxy- or C₃₋₆cycloalkyl-oxy-.

Also of interest are those compounds wherein;
n is 0;
R¹ is in the para position vis-à-vis the N-atom of the piperidine ring;
Z represents C; in particular CH or CH₂;
—X— represents C₂₋₄alkynyl, C₁₋₁₂alkyl optionally substituted with hydroxy or ‘X— represents a divalent radical of the formula (a), (b) or (c) as defined for the compounds of formula (I) hereinbefore, wherein;
—X₁— represents C₁₋₁₂alkyl, phenyl or a divalent radical of the formula (f) as defined for the compounds of formula (I) hereinbefore;
—X₂— represents C₁₋₁₂alkyl, C₁₋₄alkyloxyC₁₋₄alkyl, phenyl or a divalent radical of the formula (g) as defined for the compounds of formula (I) hereinbefore;
—X₃— represents a divalent radical of the formula (h) or (i) as defined for the compounds of formula (I) hereinbefore;
—X— represents C₂₋₄alkynyl, or X represents a divalent radical of the formula (a), (b), (c) or (j) as defined for the compounds of formula (I) hereinbefore wherein;
—X₁— represents C₁₋₁₂alkyl, phenyl or a divalent radical of the formula (e) or (f) as defined for the compounds of formula (I) hereinbefore;
—X₂— represents C₁₋₁₂alkyl or a divalent radical of formula (g) as defined for the compounds of formula (I) hereinbefore;
—X₃— represents phenyl or a divalent radical of formula (h) or (i) as defined for the compounds of formula (I) hereinbefore;
R¹ independently represents Ar¹, Ar²-carbonyl, Het² or Het¹—C₁₋₄alkyl-;
Het¹ represents a heterocycle selected from pyridinyl, pyrimidinyl, indolinyl, indolyl, benzimidazolyl, benzthiazolyl, benzisothiazolyl, benzisoxazolyl, thiazolyl, isothiazolyl or thiadiazolyl wherein said Het¹ is optionally substituted with one or where possible two or more substituents selected from the group consisting of hydroxy, halo, C₁₋₄alkyloxycarbonyl-, C₁₋₄alkyl-, C₁₋₄alkyloxy- and C₁₋₄alkyloxy-substituted with halo; in particular Het¹ represents a heterocycle selected from indolyl or pyridinyl;
Het² represents a heterocycle selected from indolyl, indolinyl, pyridinyl, pyrimidinyl, benzimidazolyl, benzoxazolyl, benzisoxazolyl, quinolinyl, quinazolinyl, quinoxalinyl, or oxodiazolyl wherein said Het² is optionally substituted with one or where possible two or more substituents selected from the group consisting of hydroxy, carbonyl, Ar⁵, halo, C₁₋₆alkyl- and C₁₋₄-alkyloxy-; in particular Het² represents indolyl, indolinyl or benzimidazolyl wherein said Het² is optionally substituted with hydroxy, carbonyl or halo, preferably substituted with hydroxy or carbonyl;
Het³ represents a heterocycle selected from benzimidazolyl, benzoxazolyl, benzisoxazolyl, benzisothiazolyl or benzthiazolyl wherein said Het³ is optionally substituted with one or where possible two or more substituents selected from the group consisting of hydroxy, halo, C₁₋₆alkyl- and C₁₋₄-alkyloxy-; in particular Het³ represents benzthiazolyl substituted with C₁₋₄alkyloxy-;
Het⁴ represents a heterocycle selected from pyrimidinyl, pyridinyl, indolinyl, indolyl, benzimidazolyl, benzoxazolyl, benzisoxazolyl, benzisothiazolyl or benzthiazolyl wherein said Het⁴ is optionally substituted with one or where possible two or more substituents selected from the group consisting of hydroxy, amino, mono or di-(C₁₋₄alkyl)amino, halo, C₁₋₆alkyl- and C₁₋₄alkyloxy-; in particular Het⁴ represents benzthiazolyl; in particular Het⁴ represents heterocycle selected from pyridinyl, indolinyl, indolyl, benzthiazolyl or benzisoxazolyl wherein said Het⁴ is optionally substituted with one or where possible two or more substituents selected from halo and C₁₋₄alkyloxy-;

In a further embodiment the compounds of the present invention consist of those compounds of formula (I) wherein n is 0, Z represents C and the R¹ substituent is in the para position vis-à-vis the N-atom of the piperidine ring. Said R¹ substituent preferably consists of phenyl or benzimidazolyl wherein said phenyl and benzimidazolyl are optionally substituted with one or more substituents selected from halo, trifluoromethyl or methyl. Of particular interest are those compounds of formula (I) wherein n is 1, R² represents hydroxy, Z represents C, R¹ represents phenyl substituted with halo and trifluoromethyl and wherein said R¹ and R² substituent are in the para position vis-à-vis the N-atom of the piperidine ring.

Another interesting group of compounds are those compounds of formula (I) wherein one or more of the following restrictions apply;
n is 1;
—X— represents C₁₋₁₂alkyl optionally substituted with hydroxyl or —X— represents a divalent radical of the formula 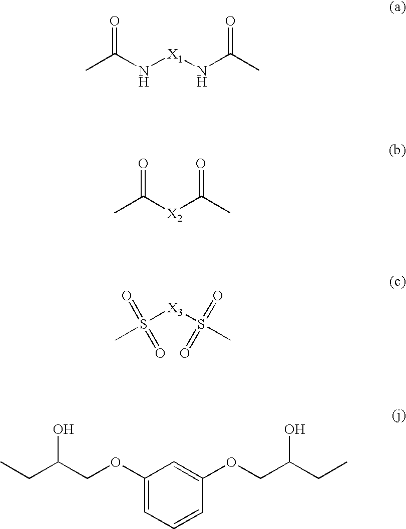
   wherein; —X₁— represents C₁₋₁₂alkyl, phenyl or the divalent radical 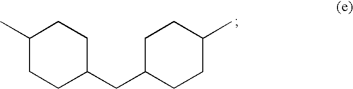
   —X₂— represents C₁₋₁₂alkyl;
   —X₃— represents 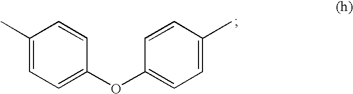
R¹ represents Ar¹;
R² represents hydroxyl;
Ar¹ represents phenyl substituted with two or more substituents selected from halo or C₁₋₄alkyl substituted with one, two or three halo substituents.

In an even further embodiment the compounds of the present invention are selected from those of formulae (A), (B), (C), (D), (E), (F), (G), (H) and (I) below:

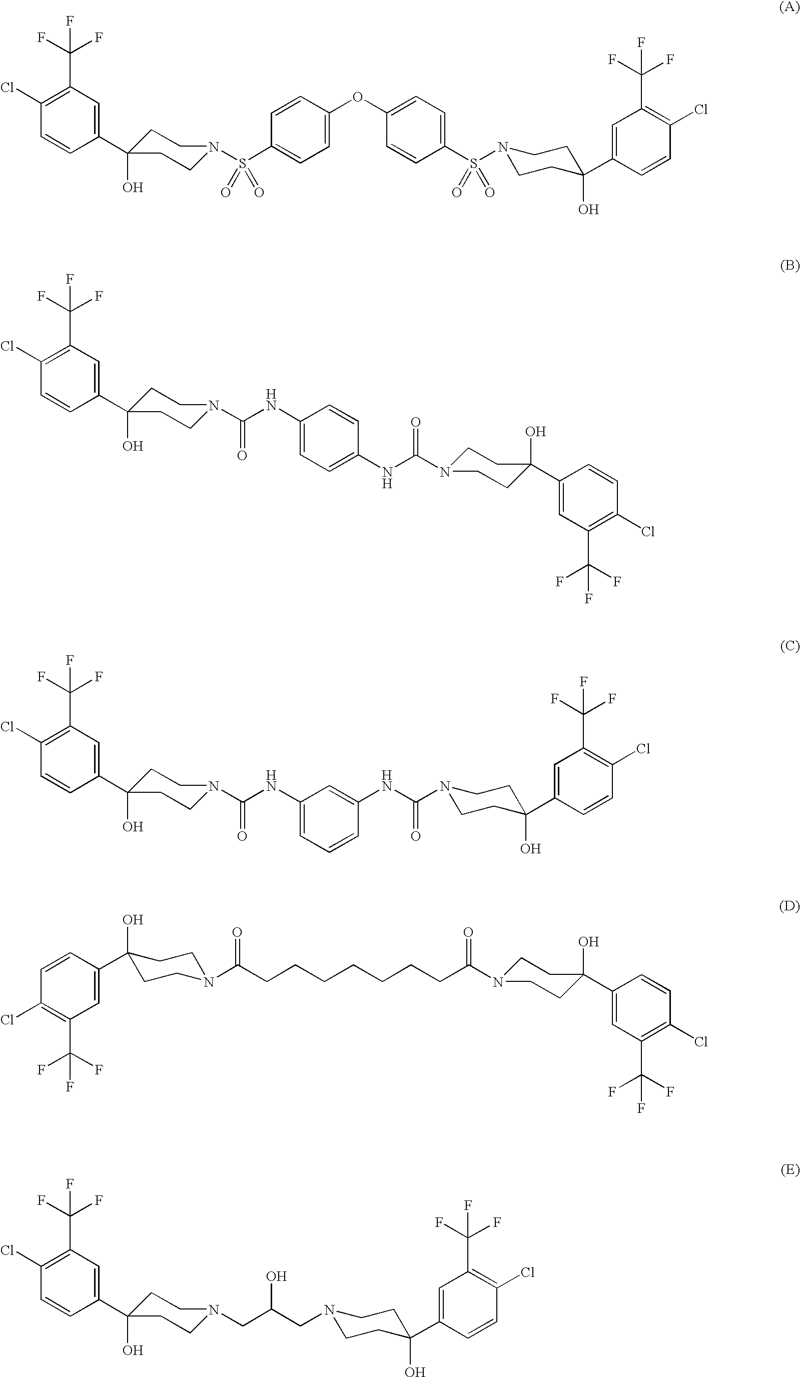

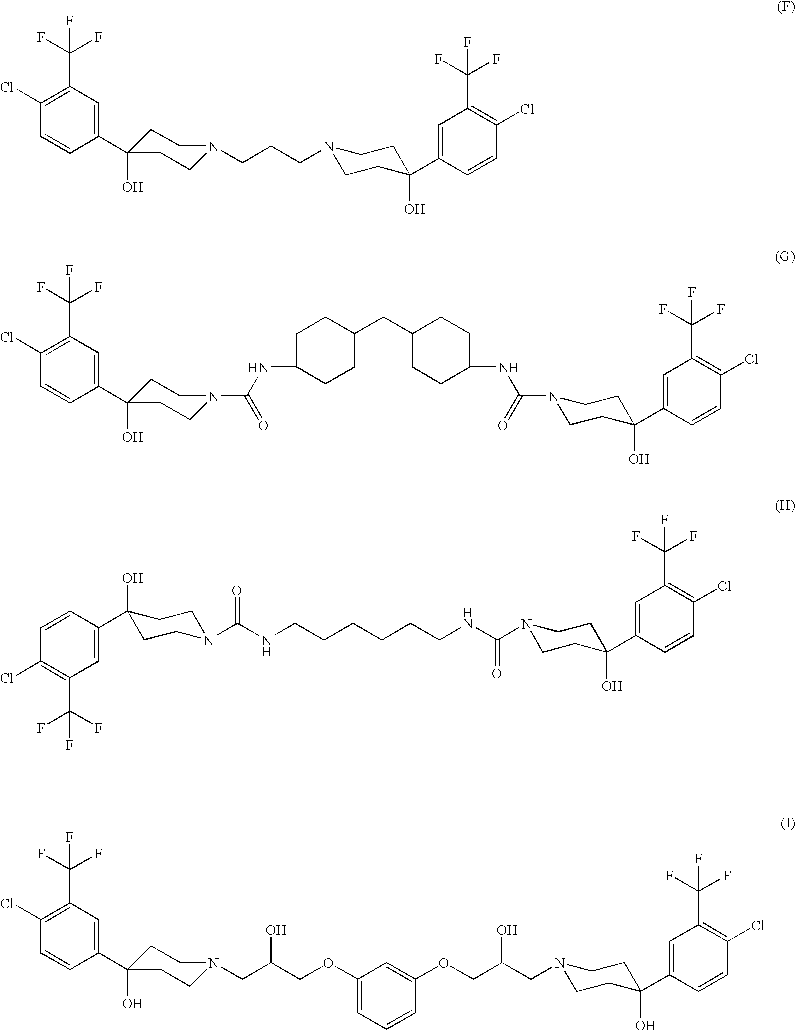

The dimeric compounds of this invention can be prepared by any of several standard synthetic processes commonly used by those skilled in the art of organic chemistry and described for instance in, “Introduction to organic chemistry” Streitweiser and Heathcock—Macmillan Publishing Co., Inc.—second edition—New York.

In general, for those compounds where X represents a C₂₋₄alkynyl or an optionally substituted C₁₋₁₂alkyl, the dimeric compounds are obtained by a nucleofilic substitution reaction between the appropriate secondary amine (i) with an alkylhalide (scheme 1) under basic reaction conditions, such as for example described in “Introduction to organic chemistry” Streitweiser and Heathcock—Macmillan Publishing Co., Inc.—second edition—New York, page 742—section 24.6.

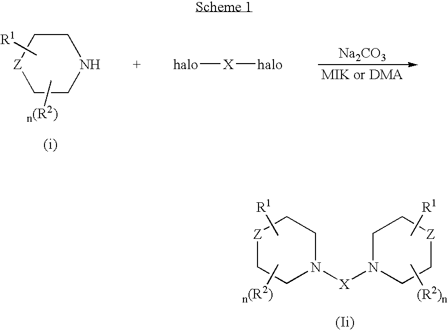

Wherein n, Z, X, R¹ and R² are defined as for the compounds of formula (I)

For those compounds where X represents a divalent radical of formula (a) the urea derivatives of formula (Iii) are prepared by reacting the appropriate secondary amine with an isocyanate of general formula (ii) under art known conditions such as for example described in “Advanced Organic Chemistry” Jerry March—John Wiley & Sons, Inc.—third edition—New York, page 802—section 6-17. 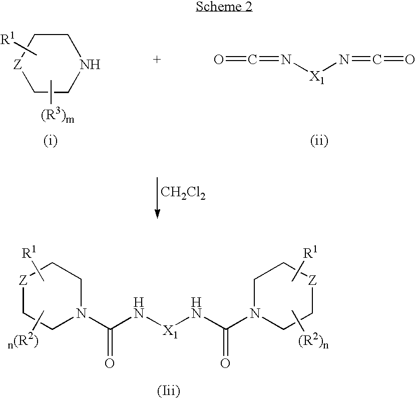
Wherein n, Z, X₁, R¹ and R² are defined as for the compounds of formula (I)

Those compounds where X represents a divalent radical of formula (b), the amide derivatives of formula (Iiii) are prepared by reacting the appropriate secondary amine with an acylhalide of general formula (iii) under art known conditions such as for example described in “Advanced Organic Chemistry” Jerry March—John Wiley & Sons, Inc.—third edition—New York, page 370—section 0-54. Alternatively the amide derivatives of formula (Iiii) are obtained by acylation of the appropriate secondary amine with an anhydride of general formula (iv) under art known conditions such as for example described in “Advanced Organic Chemistry” Jerry March—John Wiley & Sons, Inc.—third edition—New York, page 371—section 0-55, or by acylation of the appropriate secondary amine with an ester of general formula (v) under art known conditions such as for example described in “Advanced Organic Chemistry” Jerry March—John Wiley & Sons, Inc.—third edition—New York, page 375—section 0-57. 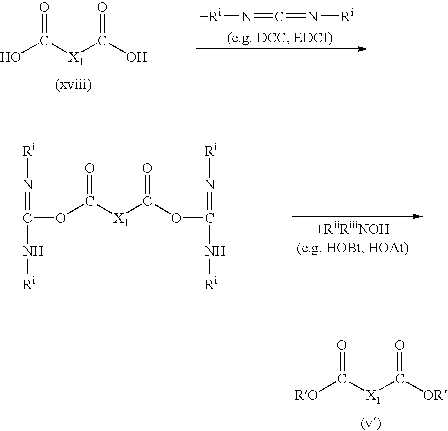
Wherein X_{I} is defined as for the compounds of formula (I) and R′ represents R^{II}R^{III}N—

In a further alternative the active ester intermediates of formula (v′) (see scheme 3) are obtained by reaction of the appropriate secondary amine with a carboxylic acid (xviii) in the presence of reagantia, i.e. coupling reagents such as for example N,N′-Dicyclohexylcarbodiimide (DCC), N-(3-Dimethylaminopropyl)-N′-ethylcarbodiimide hydrochloride (EDCI), (Benzotriazol-1-yloxy)tripyrrolidinophosphonium hexafluorophosphate (PyBOP) or O-(Benzotriazol-1-yl)-N,N,N′,N′-tetramethyluronium hexafluorophosphate (HBTU), which in a first step convert the carboxylic acid in an activated form. This reaction is preferably performed in the presence of a further hydroxylamine additive, such as 1-hydroxybenzotriazole (HOBt) or 7-aza-1-hydroxybenzotriazole (HOAt), to prevent racemization and dehydration of the carboxamide residues thus obtained. 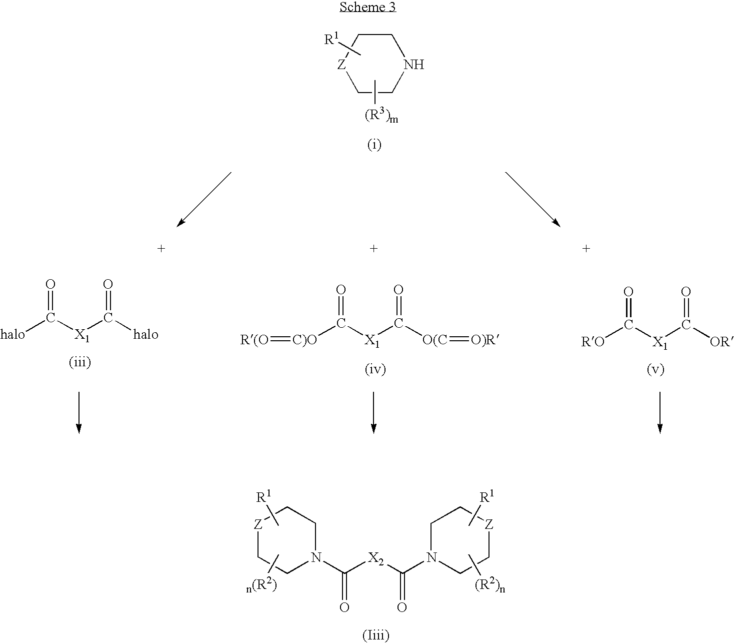
Wherein n, Z, X₂, R¹ and R² are defined as for the compounds of formula (1), R′ represents a C₁₋₄alkyl, preferably ethyl and wherein halo represents a halogen such as for example Cl, Br and I

Finally, the sulfonamide derivative of formula (Iiv) where X represents a divalent radical of formula (c) are generally prepared by a nucleophilic substitution reaction between the appropriate secondary amine and a sulfonylhalide, preferably a sulfonylchloride of general formula (vi) under art known conditions such as for example described in “Advanced Organic Chemistry” Jerry March—John Wiley & Sons, Inc.—third edition—New York, page 445—section 0-119. 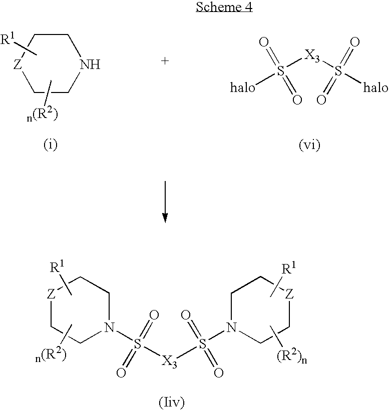
Wherein n, Z, X₃, R¹ and R² are defined as for the compounds of formula (I) and wherein halo represents a halogen such as for example Cl, Br and I, preferably Cl

The appropriate secondary amines as used hereinbefore are either commercially available or are known to, or can readily be synthesized by those of ordinary skill in the art.

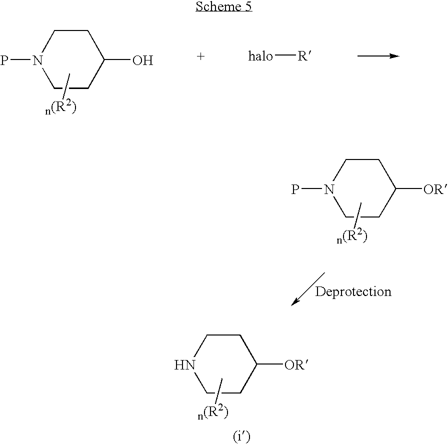

Wherein n and R² are defined as for the compounds of formula (I);

R′ represents C₁₋₄alkyl, Ar³—C₁₋₄alkyloxy- or Het⁴-oxy wherein Ar³ and Het⁴ are defined as for the compounds of formula (I); and

wherein halo represents a halogen such as for example Cl, Br and I, preferably Cl

In a particular embodiment, for those secondary amines of formula (i) wherein R¹ represents C₁₋₄alkyloxy, Ar³—C₁₋₄alkyloxy- or Het⁴-oxy hereinafter refered to as the compounds of formula (i′), said compounds are prepared departing from a protected 4-hydroxypiperidine in a nucleophilic substitution reaction with an appropriate alkylhalide under art known conditions such as for example described in “Advanced Organic Chemistry” Jerry March—John Wiley & Sons, Inc.—third edition—New York, page 3421—section 0-14 (Scheme 5).

Those secondary amines where R¹ represents NR³R⁴—C₁₋₄alkyl-, hereinafter refered to as the compounds of formula (i″) are generally prepared by acylation or alkylation of the corresponding amine using art known reaction procedures, using for example an alkyl chloride RⁱCl, an acylchloride RⁱCOCl, wherein Rⁱ represents a C₁₋₄alkyl. Further, those compounds wherein either R³ or R⁴ represents Het³ are typically obtained using art-known cyclization procedures (“Introduction to organic chemistry” Streitweiser and Heathcock—Macmillan Publishing Co., Inc.—second edition—New York, Chapter 32).

For example, for those compounds of formula i″ wherein R³ or R⁴ represents thiazolyl or benzthiazolyl the secondary amines are prepared according to reaction scheme 6. In a first step the aminomethylpiperidine of formula (vii) is converted into the intermediate of formula (ix) by reaction with an isothiocyanate of formula (viii) under art known reaction conditions (see scheme 2 above). For those intermediates where Rⁱⁱ represents hydrogen, the compounds of formula (I) are subsequently prepared by the cyclodesulfurization reaction of the thiourea derivative of formula (ix) by the reaction of (ix) with an appropriate alkyl halide (x) in an appropriate reaction-inert organic solvent, e.g., a lower alkanol such as methanol, ethanol, 2-propanol and the like. For those intermediates of formula (ix) where Rⁱⁱ does represent optionally substituted phenyl, the cyclodesulfarization reaction is carried out according to art-known procedures, such as for example using bromine in an aqueous hydrobromic acid solution.

Subsequently eliminating the protective group in the thus obtained intermediates of formula (xi) and (xi′) respectively, provides the appropriate secondary amines used as intermediates in the synthesis of the dimeric compounds of the present invention. The elimination of the protective group P in (xi, xi′) may generally be carried out following art-known procedures such as, for example, by hydrolysis in alkaline or acidic aqueous medium. 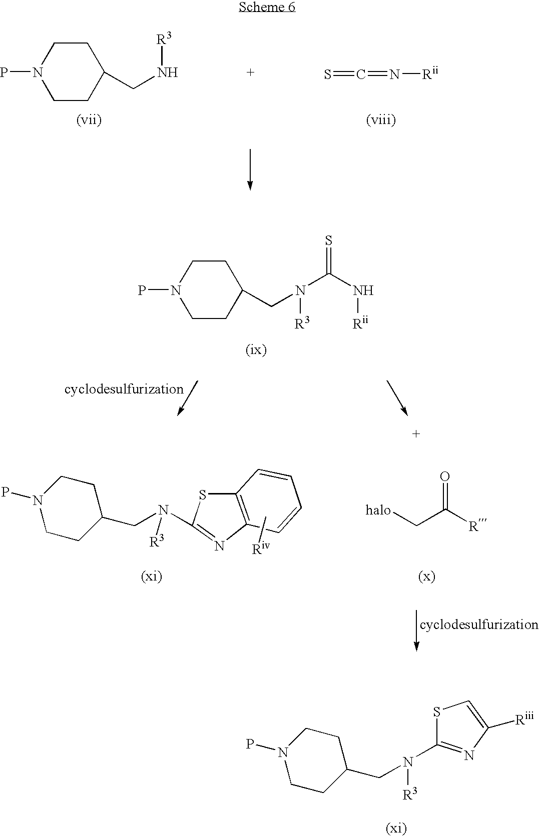
Wherein halo represents a halogen such as for example Cl, Br and I; R¹ is defined as for the compounds of formula (I); Rⁱⁱ represents hydrogen or an optionally substituted phenyl substituent; Rⁱⁱⁱ and R^{iv} each independently represent hydroxy, halo, Are, C₁₋₄alkyloxycarbonyl-, C₁₋₄alkyl-, C₁₋₄alkyloxy- or C₁₋₄alkyloxy-substituted with halo, wherein Ar⁴ is defined as for the compounds of formula (I)

Similarly the secondary amine intermediates wherein R¹ represents Het² are obtained using art known cyclization procedures. For example, for those compounds of formula (I) wherein Het² represents oxadiazolyl, the intermediates of formula (i′″) may be prepared by reacting an appropriately substituted piperidine of formula (xii) with an intermediate carboxylic ester of formula (xiii), following art known cyclization procedures and subsequently removing the protective group P, following art known procedures.

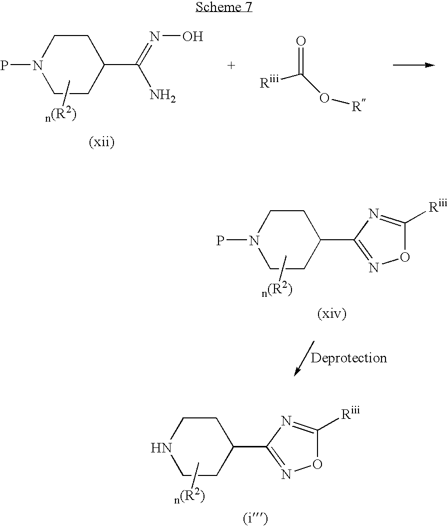

Wherein n and R² are defined as for the compounds of formula (I);

R′ represents C₁₋₄alkyl, Ar³—C₁₋₄alkyloxy- or Het⁴-oxy wherein Ar³ and Het⁴ are defined as for the compounds of formula (I); and

wherein halo represents a halogen such as for example Cl, Br and I, preferably Cl

The intermediate of formula (xii) may be prepared by reacting a cyanopiperidine derivative of formula (xv) with hydroxylamine in a reaction-inert solvent and in the presence of a strong base, such as, for example, sodium methoxide. 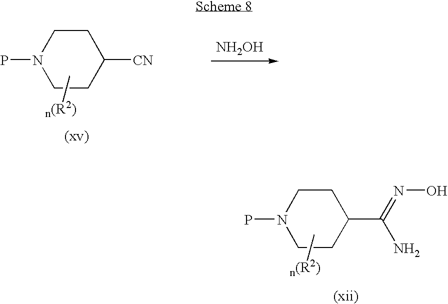
Wherein n, Z, X, R¹ and R² are defined as for the compounds of formula (I)

The intermediate carboxylic esters as used hereinbefore are generally obtained from the corresponding carboxylic acids following art-known ester formation procedures. Said corresponding carboxylic acids are known from, for example EP-0,076,530, EP-0,389,037 and EP-0,445,862.

Further examples for the synthesis of compounds of formula (I) using anyone of the above mentioned synthesis methods, are provided in the experimental part hereinafter.

Where necessary or desired, any one or more of the following further steps in any order may be performed:
(i) removing any remaining protecting group(s);
(ii) converting a compound of formula (I) or a protected form thereof into a further compound of formula (I) or a protected form thereof;
(iii) converting a compound of formula (I) or a protected form thereof into a N-oxide, a salt, a quaternary amine or a solvate of a compound of formula (I) or a protected form thereof,
(iv) converting a N-oxide, a salt, a quaternary amine or a solvate of a compound of formula (I) or a protected form thereof into a compound of formula (I) or a protected form thereof;
(v) converting a N-oxide, a salt, a quaternary amine or a solvate of a compound of formula (I) or a protected form thereof into another N-oxide, a pharmaceutically acceptable addition salt a quaternary amine or a solvate of a compound of formula (I) or a protected form thereof;

It will be appreciated by those skilled in the art that in the processes described above the functional groups of intermediate compounds may need to be blocked by protecting groups.

Functional groups which it is desirable to protect include hydroxy, amino and carboxylic acid. Suitable protecting groups for hydroxy include trialkylsilyl groups (e.g. tert-butyldimethylsilyl, tert-butyldiphenylsilyl or trimethylsilyl), benzyl and tetrahydropyranyl. Suitable protecting groups for amino include tert-butyloxycarbonyl or benzyloxycarbonyl. Suitable protecting groups for carboxylic acid include C₍₁₋₆₎alkyl or benzyl esters.

The protection and deprotection of functional groups may take place before or after a reaction step.

The use of protecting groups is fully described in ‘Protective Groups in Organic Chemistry’, edited by J W F McOmie, Plenum Press (1973), and ‘Protective Groups in Organic Synthesis’ 2^{nd} edition, T W Greene & P G M Wutz, Wiley Interscience (1991).

Additionally, the N-atoms in compounds of formula (I) can be methylated by art-known methods using CH3—I in a suitable solvent such as, for example 2-propanone, tetrahydrofuran or dimethylformamide.

The compounds of formula (I), can also be converted into each other following art-known procedures of functional group transformation of which some examples are mentioned hereinabove.

The compounds of formula (I), may also be converted to the corresponding N-oxide forms following art-known procedures for converting a trivalent nitrogen into its N-oxide form. Said N-oxidation reaction may generally be carried out by reacting the starting material of formula (I) with 3-phenyl-2-(phenylsulfonyl)oxaziridine or with an appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chlorobenzenecarboperoxoic acid, peroxoalkanoic acids, e.g. peroxoacetic acid, alkylhydroperoxides, e.g. t-butyl hydroperoxide. Suitable solvents are, for example, water, lower alkanols, e.g. ethanol and the like, hydro-carbons, e.g. toluene, ketones, e.g. 2-butanone, halogenated hydrocarbons, e.g. dichloromethane, and mixtures of such solvents.

Pure stereochemically isomeric forms of the compounds of formula (I), may be obtained by the application of art-known procedures. Diastereomers may be separated by physical methods such as selective crystallization and chromatographic techniques, e.g. counter-current distribution, liquid chromatography and the like.

Some of the compounds of formula (I), and some of the intermediates in the present invention may contain an asymmetric carbon atom. Pure stereochemically isomeric forms of said compounds and said intermediates can be obtained by the application of art-known procedures. For example, diastereoisomers can be separated by physical methods such as selective crystallization or chromatographic techniques, e.g. counter current distribution, liquid chromatography and the like methods. Enantiomers can be obtained from racemic mixtures by first converting said racemic mixtures with suitable resolving agents such as, for example, chiral acids, to mixtures of diastereomeric salts or compounds; then physically separating said mixtures of diastereomeric salts or compounds by, for example, selective crystallization or chromatographic techniques, e.g. liquid chromatography and the like methods; and finally converting said separated diastereomeric salts or compounds into the corresponding enantiomers. Pure stereochemically isomeric forms may also be obtained from the pure stereochemically isomeric forms of the appropriate intermediates and starting materials, provided that the intervening reactions occur stereospecifically.

An alternative manner of separating the enantiomeric forms of the compounds of formula (I) and intermediates involves liquid chromatography, in particular liquid chromatography using a chiral stationary phase.

Some of the intermediates and starting materials as used in the reaction procedures mentioned hereinabove are known compounds and may be commercially available or may be prepared according to art-known procedures.

The compounds of the present invention are useful because they possess pharmacological properties. They can therefore be used as medicines, in particular to treat pain, in particular post-operative pain and pathologies associated with neuronal death, such as, stroke, Alzheimer's disease, Parkinson's disease, Huntington's disease, amyotrophic lateral sclerosis, Pick's disease, fronto-temporal dementia, progressive nuclear palsy, corticobasal degeneration, cerebro-vascular dementia, multiple system atrophy, argyrophilic grain dementia, and other tauopathies. Further conditions involving neurodegenerative processes are for instance, age-related macular degeneration, narcolepsy, motor neuron diseases, prion diseases, traumatic nerve injury and repair, and multiple sclerosis.

As described in the experimental part hereinafter, the neurotrophic activity of the present compounds on p75 mediated neuronal death has been demonstrated in vitro, in an assay that determines the survival effect of the compounds on chick DRG neurons using the neurotrophic factor NGF as internal reference. This assay is based on a fluorimetric Calcein-AM measurement and addresses the functional response of neurons as a quantitative measure of survival.

Accordingly, the present invention provides the compounds of formula (I) and their pharmaceutically acceptable N-oxides, addition salts, quaternary amines and stereochemically isomeric forms for use in therapy. More particular in the treatment or prevention of neurodegenerative mediated disorders. The compounds of formula (I), and their pharmaceutically acceptable N-oxides, addition salts, quaternary amines and the stereochemically isomeric forms may hereinafter be referred to as compounds according to the invention.

In view of the utility of the compounds according to the invention, there is provided a method for the treatment of an animal, for example, a mammal including humans, suffering from a neurodegenerative disorder such as stroke, Alzheimer's disease, ALS, epilepsy, SCI, MS, MND and other neurodegenerative diseases as mentioned hereinbefore, which comprises administering an effective amount of a compound according to the present invention. Said method comprising the systemic or topical administration of an effective amount of a compound according to the invention, to warm-blooded animals, including humans.

It is thus an object of the present invention to provide a compound according to the present invention for use as a medicine. In particular to use the compound according to the present invention in the manufacture of a medicament for treating pathologies associated with neuronal death such as for example, stroke, Alzheimer's disease, ALS, epilepsy, SCI, MS, MND and other neurodegenerative diseases as mentioned hereinbefore.

In yet a further aspect, the present invention provides the use of the compounds according to the invention in the manufacture of a medicament for treating any of the aforementioned neurodegenerative disorders or indications.

The amount of a compound according to the present invention, also referred to here as the active ingredient, which is required to achieve a therapeutical effect will be, of coursekvary with the particular compound, the route of administration, the age-and condition of the recipient, and the particular disorder or disease being treated. A suitable daily dose would be from 0.001 mg/kg to 500 mg/kg body weight, in particular from 0.005 mg/kg to 100 mg/kg body weight. A method of treatment may also include administering the active ingredient on a regimen of between one and four intakes per day.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of this invention may be prepared by any methods well known in the art of pharmacy, for example, using methods such as those described in Gennaro et al. Remington's Pharmaceutical Sciences (18^{th} ed., Mack Publishing Company, 1990, see especially Part 8: Pharmaceutical preparations and their Manufacture). A therapeutically effective amount of the particular compound, in base form or addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous, or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions: or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment. As appropriate compositions for topical application there may be cited all compositions usuallyemployed for topically administering drugs e.g. creams, gellies, dressings, shampoos, tinctures, pastes, ointments, salves, powders and the like. Application of said compositions may be by aerosol, e.g. with a propellant such as nitrogen, carbon dioxide, a freon, or without a propellant such as a pump spray, drops, lotions, or a semisolid such as a thickened composition which can be applied by a swab. In particular, semisolid compositions such as salves, creams, gellies, ointments and the like will conveniently be used.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

In order to enhance the solubility and/or the stability of the compounds of formula (I) in pharmaceutical compositions, it can be advantageous to employ α-, β- or γ-cyclo-dextrins or their derivatives. Also co-solvents such as alcohols may improve the solubility and/or the stability of the compounds of formula (I) in pharmaceutical compositions. In the preparation of aqueous compositions, addition salts of the subject compounds are obviously more suitable due to their increased water solubility.

Experimental Part

Hereinafter, the term ‘RT’ means room temperature, ‘MIK’ means 4-methyl-2-pentanone, ‘THF’ means tetrahydrofuran, ‘DIPE’ means diisopropyl ether, ‘DMSO’ means dimethylsulfoxide.

### A. PREPARATION OF THE INTERMEDIATES

### EXAMPLE A1

### a) Preparation of

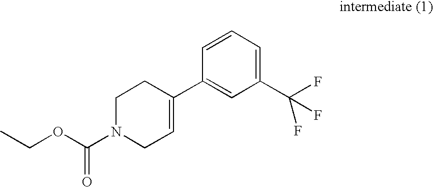

Chlorocarbonic acid, ethyl ester (0.25 mol) was added dropwise at 20° C. to a mixture of 1,2,3,6-tetrahydro-4-[3-(trifluoromethyl)phenyl]pyridine (0.2 mol) and sodium carbonate (0.21 mol) in dichloromethane (600 ml), while the mixture was cooled now and then. The mixture was stirred for 4 hours. Water was added and the mixture was separated into its layers. The organic layer was dried, filtered and the solvent was evaporated, yielding 56 g (93%) of intermediate (1).

### b) Preparation of

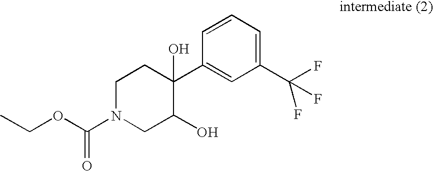

A mixture of intermediate (1) (0.19 mol) and sodium hydrogen carbonate (0.25 mol) in dichioromethane (500 ml) was cooled to 50C. 3-Chlorobenzenecarboperoxoic acid (0.25 mol) was added quickly. The mixture was stirred at 20° C. overnight, then filtered, washed twice with a saturated NaHCO₃ solution, a saturated Na₂SO₃ solution, a diluted HCl solution, water, a NaOH 3% solution and water. The organic layer was dried, filtered and the solvent was evaporated. The product was used without further purification, yielding 4.5 g (71%) of intermediate (2).

### c) Preparation of

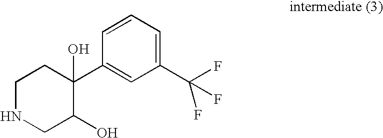

A mixture of intermediate (2) (0.14 mol) and potassium hydroxide (1.2 mol) in 2-propanol (11) was stirred and refluxed for 6 hours. The solvent was evaporated. Ice water was added. The mixture was extracted with dichloromethane. The organic layer was separated, dried, filtered and the solvent was evaporated, yielding 20 g (55%) of intermediate (3).

### EXAMPLE A2

### a) Preparation of

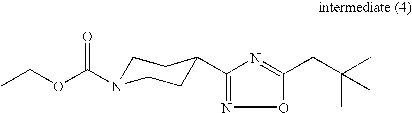

A mixture of 4-[(hydroxyamino)iminomethyl]-1-piperidinecarboxylic acid, ethyl ester [182808-27-1] (0.079 mol) and molecular sieves (21 g) in 1,4-dioxane (400 ml) was stirred at 10° C., under N₂ flow. Sodium hydride (60%) (0.085 mol) was added portionwise over 30 minutes (foaming!). The mixture was stirred for 30 minutes at room temperature. A solution of 3,3-dimethylbutanoic acid, methyl ester in 1,4-dioxane (100 ml) was added and the resulting reaction mixture was stirred and refluxed for 5 hours, then overnight at room temperature. Water (200 ml) was added. CH₂Cl₂ was added and the biphasic mixture was filtered over dicalite. The layers of the filtrate were separated. The organic layer was dried, filtered, and the solvent evaporated. The residue was purified over silica gel on a glass filter (eluent: CH₂Cl₂/CH₃OH: 97/3). The pure fractions were collected and the solvent was evaporated, yielding 16.2 g (70%) of intermediate (4).

### b) Preparation of

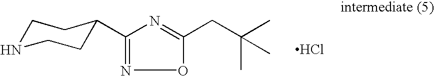

A mixture of intermediate (4) (0.0519 mol) and potassium hydroxide (0.5 mol) in 2-propanol (750 ml) was stirred and refluxed for 5 hours, then overnight at room temperature. The solvent was evaporated. The residue was stirred in water and this mixture was extracted three times with dichloromethane. The separated organic layer was dried, filtered, and the solvent evaporated. The residue was purified over silica gel on a glass filter (eluent: CH₂Cl/CH₃OH/(CH₃OH/NH₃) 90/5/5). The pure fractions were collected and the solvent was evaporated, yielding 8.6 g (75.5%) of product. Part (6.4 g) of this fraction was dissolved in 2-propanol (50 ml) and converted into the hydrochloric acid salt (1: 1) with HCl/2-propanol. The precipitate was filtered off, washed with DIPE, and dried, yielding 6.1 g of intermediate (5), isolated as its hydrochloric acid salt; mp. 212.2° C.

### EXAMPLE A3

### a) Preparation of

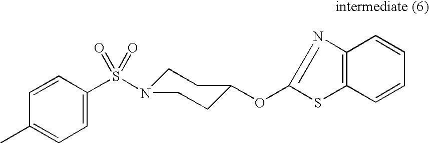

This reaction was performed under N₂ flow. Sodium hydride (50%) (0.04 mol) was added portionwise to a solution of 1-[(4-methylphenyl)sulfonyl]-4-piperidinol (0.04 mol) in DMF (150 ml). The mixture was stirred for one hour at room temperature. A (fuming) solution of 2-chlorobenzothiazole (0.04 mol) in DMF (50 ml) was added dropwise (exothermic reaction!) and the resultant reaction mixture was stirred over the l0 weekend at room temperature. The mixture was poured out into ice-water and the resulting precipitate was filtered off, washed with water and petroleum ether, then dissolved in dichloromethane. The organic solution was dried, filtered and the solvent evaporated. The residue was crystallized from 2-propanol, filtered off and dried, yielding 12.8 g (82.3%) of intermediate (6); m.p. 150.4° C. (EA: C: −0.28, H: +0.00, N: −0.11, O: −0.14, S: −0.29).

### b) Preparation of

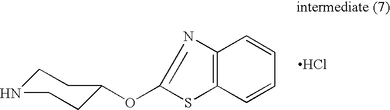

A mixture of intermediate (6)(0.03 mol), DMF (100 ml), 1,2-diaminoethane (60 ml) and N,N,N-triethylethanaminium bromide (4 g) was detosylated electrochemically (Hg cathode, C anode). Upon reaction completion, the reaction mixture was poured out into ice-water. The mixture was extracted with dichloromethane. The organic layer was separated, dried, filtered and the solvent evaporated, yielding 7.5 g of residue. Part (1 g) of the residue was dissolved in 2-propanol and converted into the hydrochloric acid salt (1:1) with HCl/2-propanol. The precipitate was filtered off and dried, yielding 1 g (86.5%) of intermediate (7); m.p. 228.9° C. (EA: C: −0.22, H: +0.20, N: −0.21, S: −0.04; Cl: −0.64).

### EXAMPLE A4

### a) Preparation of

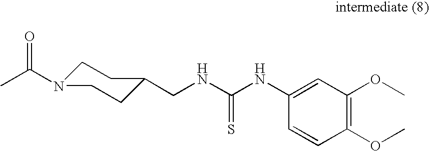

A solution of 4-isothiocyanato-1,2-dimethoxybenzene [33904-04-0] (0.16 mol) in DIPE was added to a mixture of 1 -acetyl-4-piperidinemethanamine [77445-06-8] (0.16 mol) in acetonitrile (300 ml). The mixture was stirred at room temperature for 3 hours. The solvent was evaporated. The residue was taken up in CHCl₃, washed with water, dried (MgSO₄), filtered and the solvent was evaporated. The residue was crystallized from CH₃OH/DIPE. The precipitate was filtered off and dried, yielding 25.5 g (44.5%) of intermediate (8); m.p. 161.3° C.

### b) Preparation of

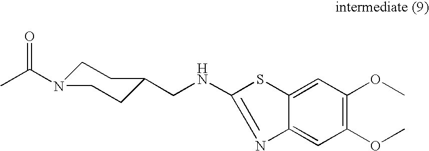

A mixture of intermediate (8) (0.073 mol) and bromine (0.073 mol) in tetrachloromethane (250 ml) was stirred and refluxed for 3 hours. The mixture was cooled. The precipitate was filtered off, taken up in water, alkalized with NaOH and extracted with MIK. The organic layer was separated, dried (MgSO₄), filtered and the solvent was evaporated, yielding 18 g (70.6%) of intermediate (9).

### c) Preparation of

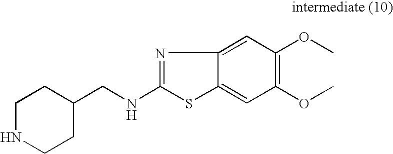

A mixture of intermediate (9)(0.05 mol) and potassium hydroxide (0.5 mol) in 2-propanol (300 ml) was stirred and refluxed overnight. The solvent was evaporated. The residue was taken up in water and the organic solvent was evaporated. The concentrate was extracted with dichloromethane. The organic layer was separated, dried (MgSO₄), filtered and the solvent was evaporated. The residue was crystallized from CH₃CN. The precipitate was filtered off and dried, yielding 7.5 g (49%) of intermediate (10); m.p. 184° C.

### EXAMPLE A5

### a) Preparation of

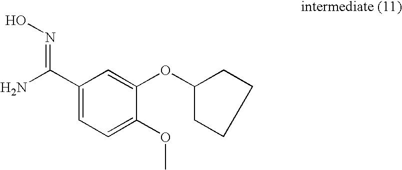

A mixture of hydroxylamine monohydrochloride(0.72 mol) in ethanol (600 ml) was stirred at room temperature. A solution of sodium carbonate (0.36 mol) in water (600 ml) was added dropwise. A solution of 3-(cyclopentyloxy)-4-methoxybenzonitrile [159783-16-1] (0.36 mol) in ethanol (600 ml) was added and the reaction mixture was stirred and refluxed for 3 hours. More hydroxylamine monohydrochloride (5 g) was added. More sodium carbonate (2 g) was added and the reaction mixture was stirred and refluxed for 30 minutes. The solvent was evaporated. The residue was stirred in ice water (500 ml) and this mixture was extracted with dichloromethane. The separated organic layer was dried (MgSO₄), filtered and the solvent evaporated, yielding 91.9 g. The residue was purified over silica gel on a glass filter (eluent: CH₂Cl₂/CH₃OH 98/2). The desired fractions were collected and the solvent was evaporated, yielding 27 g of fraction 1. This fraction (27 g) was stirred in DIPE, filtered off, washed with DIPE and dried (vacuum; 60° C.), yielding 18.5 g of intermediate (11).

### b) Preparation of

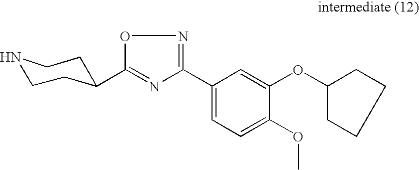

Reaction under N₂ flow. A suspension of intermediate (11) (0.006 mol) in THF (dry) (20 ml) was stirred at 0° C. Sodium hydride (60%) (0.006 mol) was added portionwise. The mixture was stirred for 15 minutes at 0° C., then for 90 minutes at reflux ,.temperature. A solution of 4-piperidinecarboxylic acid, ethyl ester (0.006 mol) in THF (dry) (10 mi) was added. The reaction mixture was stirred and refluxed for two nights. The solvent was evaporated. Dioxane (25 ml) was added. Molecular sieves (7 g) were added and the reaction mixture was stirred and refluxed for 2 hours. More 4-piperidinecarboxylic acid, ethyl ester (0.94 g) was added. The reaction mixture was stirred and refluxed overnight. The mixture was cooled, filtered and washed well with dioxane. The filtrate was evaporated. The residue (2.3 g) was purified over silica gel on a glass filter (eluent: CH₂Cl₂/CH₃OH/(CH₃OH/NH₃) 95/2.5/2.5, upgrading to 90/5/5). The desired fractions were collected and the solvent was evaporated. The residue (1.9 g) was crystallized from CH₃CN (10 ml). The precipitate was filtered off, washed with DIPE and dried (vacuum; 50° C.), yielding 0.75 g (36.4%) of intermediate (12); m.p. 96.5° C.

### B. PREPARATION OF THE COMPOUNDS

### EXAMPLE B1

A mixture of (4-fluorophenyl)-4-piperidinylmethanone (0.01 mol), 1,4-dichloro-2-butyne (0.005 mol) and sodium carbonate (1 g) in MIK (20 ml) was stirred overnight at 100° C. The reaction mixture was washed with water, and the organic solvent was evaporated. The residue was purified by HPLC over Kromasil silica gel (200 g, 100 Å, 5 μm) (eluent: CH₂Cl₂/(CH₂Cl₂/CH₃OH 90/10)/CH₃OH (0 min) 100/0/0, (34 min) 0/100/0, (40 min) 50/0/50, (43 min) 0/0/100, (46.6-60 min) 100/0/0). The pure fractions were collected and the solvent was evaporated, yielding 0.75 g of product. This fraction was dried, yielding 0.558 g of of compound 1.

### EXAMPLE B2

The catalyst palladium on activated carbon (0.100 g) was suspended in methanol (2 ml), under nitrogen. A thiophene solution in DIPE (1 ml; 0.4% solution in DIPE) was added along with a solution of dodecanedial (0.0005 mol) in THF (2 ml) and a solution of 2,3-dihydro-1-(4-piperidinyl)-1H-indole (0.001 mol) in methanol (2 ml). Hydrogenation was done at 50° C. (uptake of hydrogen (2 equiv.)). The catalyst was filtered off, the filtrate evaporated and purified by high-performance liquid chromatography over Kromasil Spherical underivated silica gel (55 g, 60 Å, 5 μm; eluent: CH₂Cl₂/(CH₂Cl₂/CH₃OH 9/1)/CH₃0H (0 min) 100/0/0, (10.50 min) 0/100/0, (12.50 min) 50/0/50, (14.00 min) 0/0/100, (15.01-20.00 min) 100/0/0). The desired fractions were collected and the solvent was evaporated, yielding 0.025 g of compound 2. This compound (0.025 g) was dissolved in DMSO (2.19 ml) and used for pharmacological tests.

### EXAMPLE B3

A mixture of 1,6-diisocyanatohexane (0.00021 mol) and intenriediate (3) (0.00042 mol) in THF (5 ml) was stirred overnight at 40° C. The reaction mixture was evaporated and purified by column chromatography over silica gel (eluent: CH₂Cl₂/CH₃OH 90/10). The pure fractions were collected and the solvent was evaporated, yielding 0.063 g of compound 3.

### EXAMPLE B4

A solution of intermediate (5) (0.0005 mol) in dichloromethane (2 ml) was mixed with a solution of N,N-diethylethanamine (0.0012 mol) in dichloromethane (2 ml). The mixture was cooled on an ice-bath. This mixture was treated dropwise with a solution of heptanedioyl dichloride (0.00026 mol) in dichloromethane (2 ml). The reaction mixture was stirred overnight at room temperature. The reaction mixture was evaporated and purified by high-performance liquid chromatography over Kromasil Spherical Silica (55 g, 60 Å, 5 μm; eluent: CH₂Cl₂/(CH₂Cl₂/CH₃OH 9/1)/CH₃OH (0 min) 100/0/0, (10.31 min) 0/100/0, (10.32 min) 50/0/50, (13.02 min) 0/0/100, (13.33-18.32 min) 100/0/0). The desired fractions were collected and the solvent was evaporated, yielding 0.100 g compound 4. This compound (0.100 g) was dissolved in DMSO (8.76 ml) and used for pharmacological tests.

### EXAMPLE B5

A mixture of 4-(3-pyridinyl)-4-piperidinol (0.00040 mol) in dichloromethane (4 ml) with N,N-diethylethanamine (1.5 ml; 5% in CH₂Cl₂) was stirred. Diphenylmethane-4,4′-disulfonyl chloride (0.00020 mol) was added and the reaction mixture was stirred overnight at room temperature. The reaction mixture was evaporated and purified by column chromatography. The pure fractions were collected and the solvent was evaporated, yielding 0.005 g of compound 5.

Table F-1 lists the compounds that were prepared according to one of the above Examples.

TABLE F-1

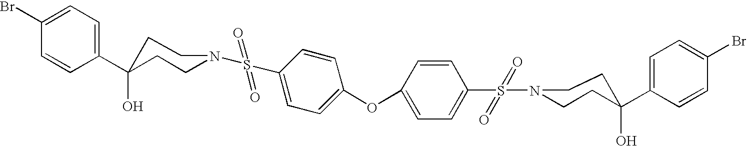

Co. No. 6; Ex. B.5

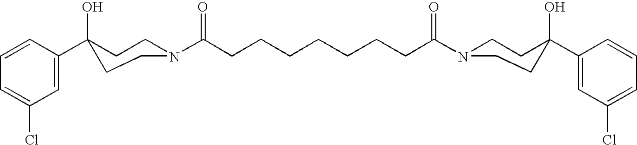

Co. No. 7; Ex. B.4

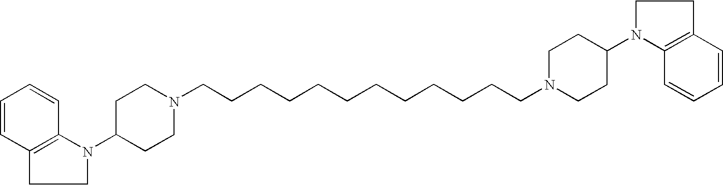

Co. No. 2; Ex. B.2

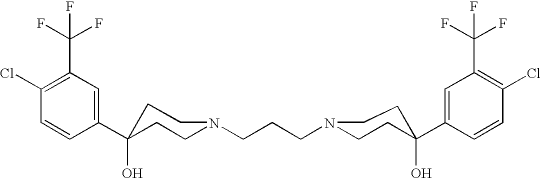

Co. No. 8; Ex. B.1

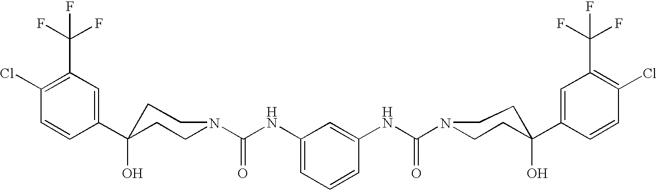

Co. No. 9; Ex. B.3

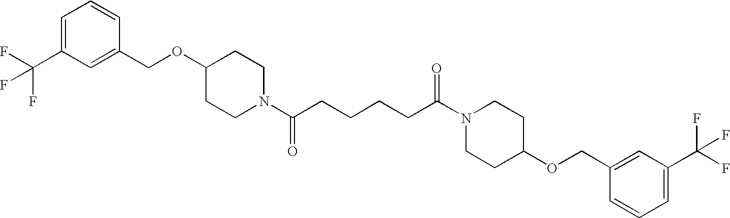

Co. No. 10; Ex. B.4

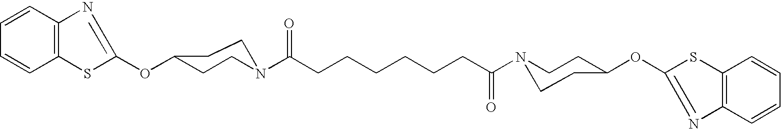

Co. No. 11; Ex. B.4

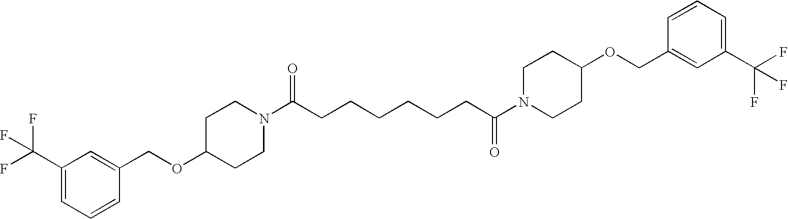

Co. No. 12; Ex. B.4

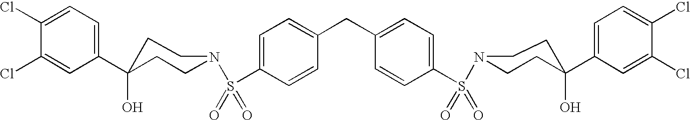

Co. No. 13; Ex. B.5

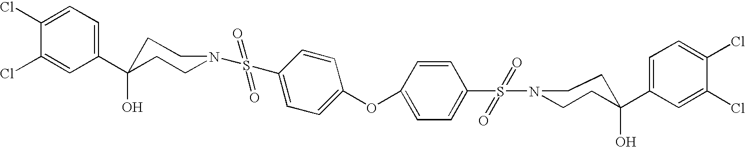

Co. No. 14; Ex. B.5

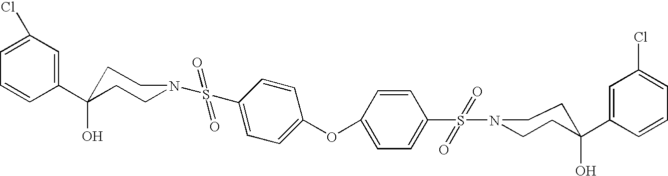

Co. No. 15; Ex. B.5

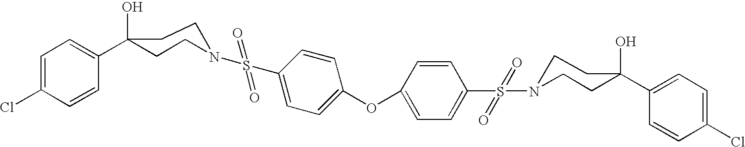

Co. No. 16; Ex. B.5

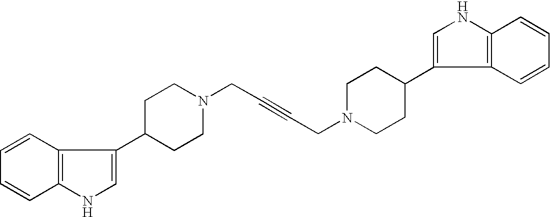

Co. No. 17; Ex. B.1

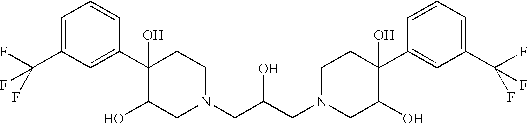

Co. No. 18; Ex. B.1

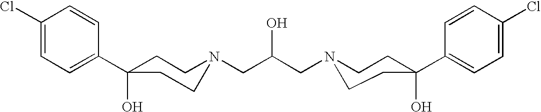

Co. No. 19; Ex. B.1

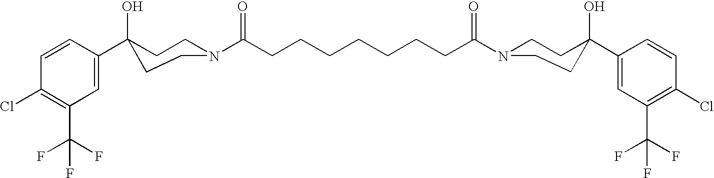

Co. No. 20; Ex. B.4

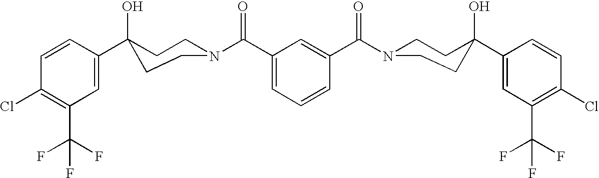

Co. No. 21; Ex. B.4

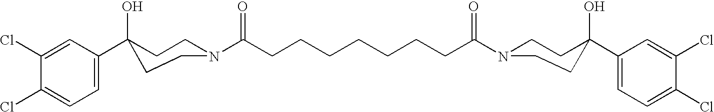

Co. No. 22; Ex. B.4

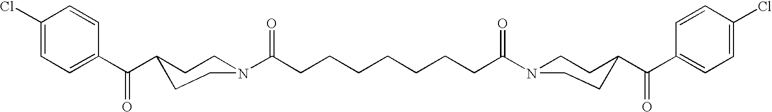

Co. No. 23; Ex. B.4

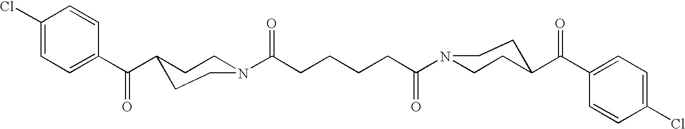

Co. No. 24; Ex. B.4

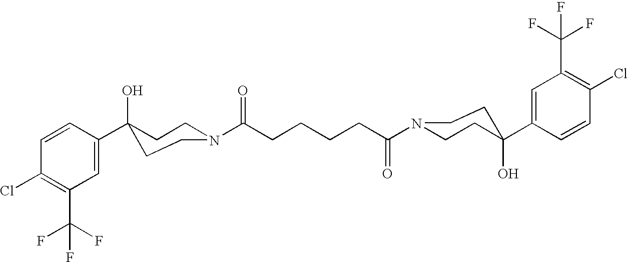

Co. No. 25; Ex. B.4

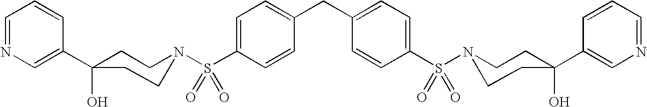

Co. No. 5; Ex. B.5

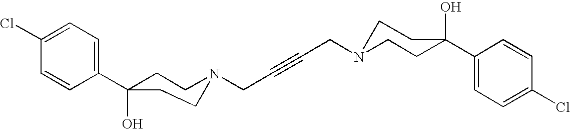

Co. No. 26; Ex. B.1

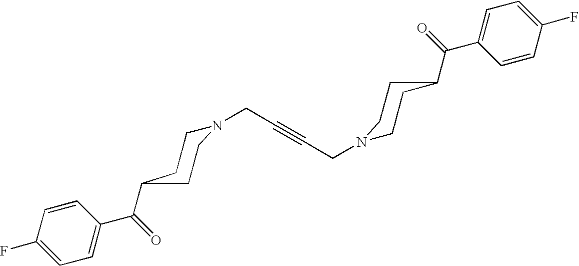

Co. No. 1; Ex. B.1

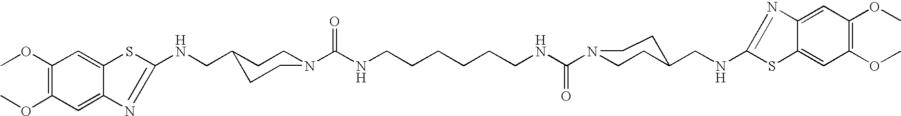

Co. No. 27; Ex. B.3

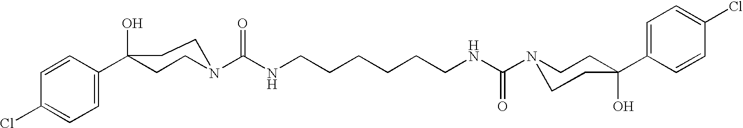

Co. No. 28; Ex. B.3

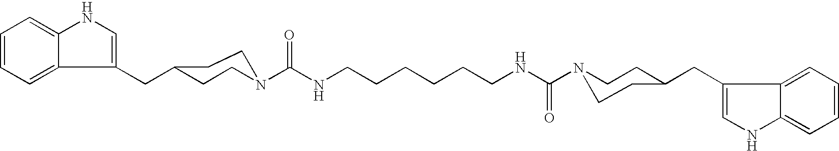

Co. No. 29; Ex. B.3

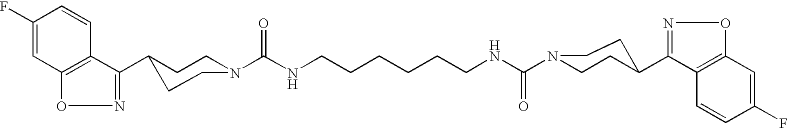

Co. No. 30; Ex. B.3

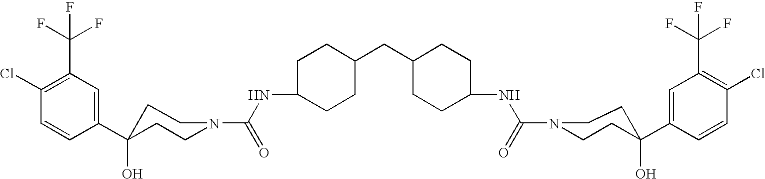

Co. No. 31; Ex. B.3

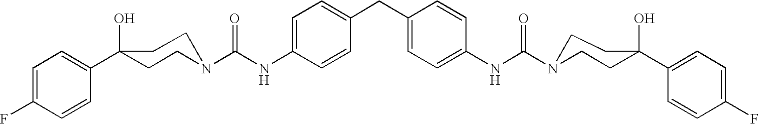

Co. No. 32; Ex. B.3

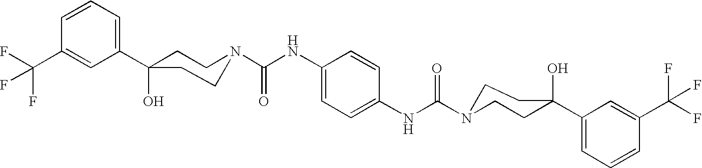

Co. No. 33; Ex. B.3

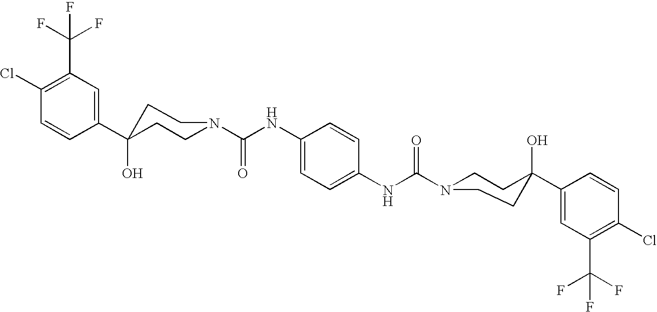

Co. No. 34; Ex. B.3

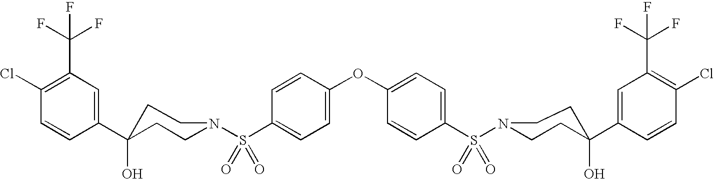

Co. No. 35; Ex. B.5

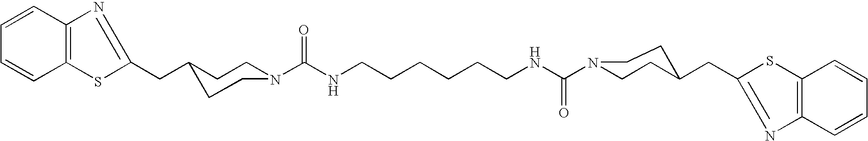

Co. No. 36; Ex. B.3

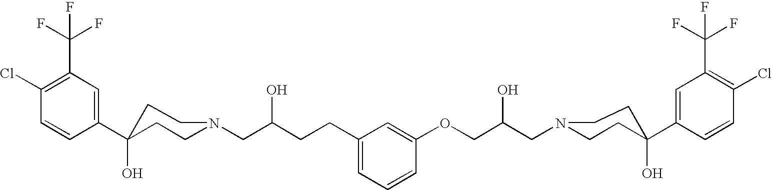

Co. No. 37; Ex. B.1; mp. 146° C.

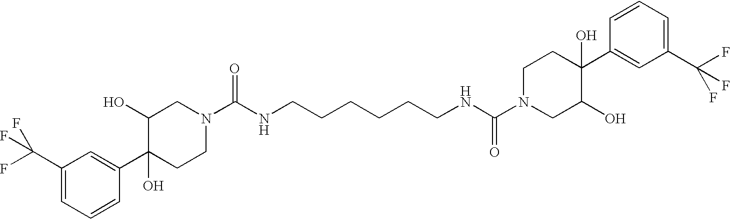

Co. No. 3; Ex. B.3

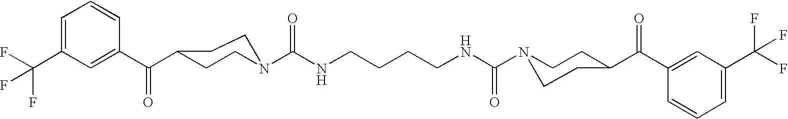

Co. No. 38; Ex. B.3

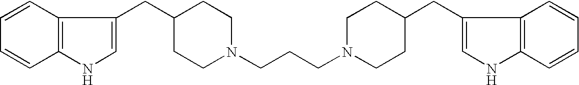

Co. No. 39; Ex. B.1

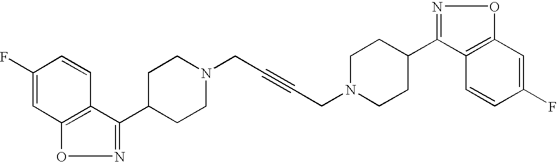

Co. No. 40; Ex. B.1

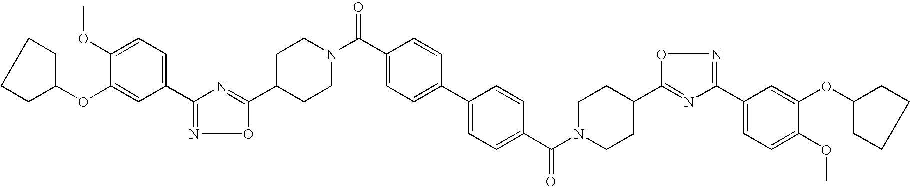

Co. No. 41; Ex. B.4

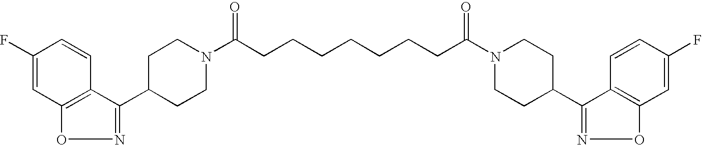

Co. No. 42; Ex. B.4

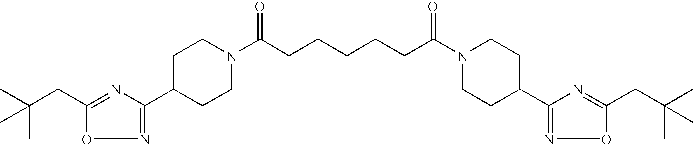

Co. No. 4; Ex. B.4

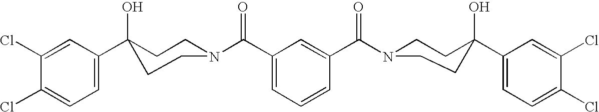

Co. No. 43; Ex. B.4

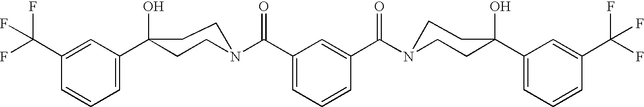

Co. No. 44; Ex. B.4

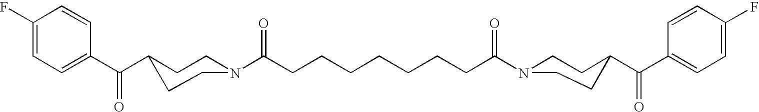

Co. No. 45; Ex. B.4

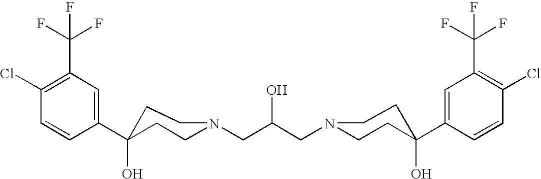

Co. No. 46; Ex. B.1

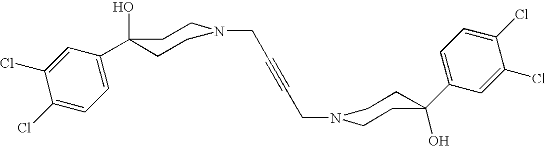

Co. No. 47; Ex. B.1

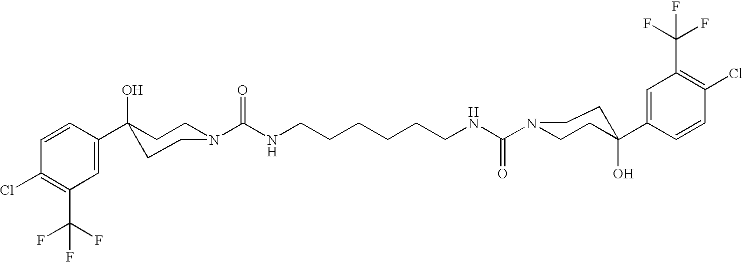

Co. No. 48; Ex. B.3

### C. PHARMACOLOGICAL EXAMPLES

### EXAMPLE C.1: NEURONAL VIABILITY ASSAY

Primary Culture of Chicken Dorsal Root Ganglion Neurons

Dorsal root ganglia were dissected from White Leghorn chick embryos at embryonic day 10 as described previously (Skaper S. D. and Varon S. (1986) Brain Research 389, 39-46). The ganglia were trypsinised and dissociated by mild trituration in a HBSS buffer supplemented with 0.6% glucose and 0.08% trypsin. To remove non-neuronal lo cells by differential attachment to culture plastic, the ganglionic cell suspension was diluted to 2.5×10⁵ cells/ml and seeded on tissue culture plastic dishes at 10 ml per 100 mm dish. After 2 h preplating, unattached neurons were collected and resuspended into Basal Eagle Medium containing 10% FCS. To remove cell aggregates, the cell suspension was passed through a nylon mesh (50 μM) pore diameter. Neuron-enriched cell suspension was plated at 5×10⁴ cells/ml into poly-L-ornithine (100 μg/ml) and laminine (1 μg/ml) coated multiwell 96 plates. Compounds were dissolved in dimethyl sulfoxide and kept as a stock at −20° C. NGF and compounds were diluted in the culture medium and added to the cells immediately after plating. The final concentration of dimethyl sulfoxide in the test medium was 0.1%. After two days of incubation, neuronal viability was assessed with calcein-AM.

Neuronal Viability Assay Using Calcein-AM

Neuronal viability assay using calcein AM was performed as previously described (Bozyczko-Coyne D., McKenna B. W., Connors T. J., and Neff N. T. (1993) Journal of Neuroscience Methods 50, 205-216). For the assay, calcein-AM was diluted in PBS to the final concentration (1 μM). For each experiment an aliquot of calcein-AM (1 mg/ml in DMSO stored at −20° C.) was thawed immediately before use. The medium was removed from the wells and replaced with the calcein-AM solution. Assay plates were incubated for 1 h at 37° C. in a humidified CO₂ incubator. Following the incubation, reading was done in a Cytofluor II at an excitation wavelength of 485 nm and an emission wavelength of 530 nm. Each plate had control wells with no neurotrophic factor added (0% survival) and wells with 10 ng/ml NGF (100% survival).

The drugs to be tested were taken from a stock solution and tested at a final concentration ranging from −10⁻⁵M to 3.10⁻⁹M. From the thus obtained dose response curves, the pIC50 value was calculated and scored as follows; Score 1=pIC50 value <6, Score 2=pIC50 value in the range of 6 to 8, Score 3 =pIC50 value>8. Some of the thus obtained results are summarized in the table below. (in this table NT stands for Not Tested).

| | [C1] DRG |
|---|---|
| Compound | assay |
| Number | SCORE |
| 6 | 1 |
| 7 | 1 |
| 2 | 2 |
| 10 | 2 |
| 11 | 2 |
| 12 | 2 |
| 13 | 1 |
| 14 | 2 |
| 15 | 2 |
| 16 | 2 |
| 17 | 2 |
| 18 | 2 |
| 19 | 2 |
| 20 | 2 |
| 21 | 2 |
| 22 | 1 |
| 23 | 2 |
| 24 | 2 |
| 25 | 1 |
| 5 | 2 |
| 26 | 3 |
| 1 | 3 |
| 27 | 2 |
| 28 | 2 |
| 29 | 2 |
| 30 | 2 |
| 32 | 1 |
| 33 | 2 |
| 34 | 2 |
| 36 | 2 |
| 3 | 2 |
| 38 | 2 |
| 39 | 2 |
| 40 | 2 |
| 41 | 2 |
| 42 | 2 |
| 4 | 1 |
| 43 | 1 |
| 44 | 1 |
| 45 | 1 |
| 47 | 2 |
| 48 | 2 |

### D. COMPOSITION EXAMPLES

The following formulations exemplify typical pharmaceutical compositions suitable for systemic or topical administration to animal and human subjects in accordance with the present invention.

“Active ingredient” (A.I.) as used throughout these examples relates to a compound of 15 formula (I) or a pharmaceutically acceptable addition salt thereof.

### EXAMPLE D.1: FILM-COATED TABLETS

### Preparation of Tablet Core

A mixture of A.I. (100 g), lactose (570 g) and starch (200 g) was mixed well and thereafter humidified with a solution of sodium dodecyl sulfate (5 g) and polyvinyl-pyrrolidone (10 g) in about 200 ml of water. The wet powder mixture was sieved, dried and sieved again. Then there was added microcrystalline cellulose (100 g) and hydrogenated vegetable oil (15 g). The whole was mixed well and compressed into tablets, giving 10,000 tablets, each comprising 10 mg of the active ingredient.

Coating

To a solution of methyl cellulose (10 g) in denaturated ethanol (75 ml) there was added a solution of ethyl cellulose (5 g) in CH₂Cl₂ (150 ml). Then there were added CH₂Cl₂ (75 ml) and 1,2,3-propanetriol (2.5 ml). Polyethylene glycol (10 g) was molten and dissolved in dicbloromethane (75 ml). The latter solution was added to the former and then there were added magnesium octadecanoate (2.5 g), polyvinyl-pyrrolidone (5 g) and concentrated color suspension (30 ml) and the whole was homogenated. The tablet cores were coated with the thus obtained mixture in a coating apparatus.

1. A compound having the formula 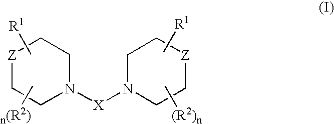
the N-oxide forms, the pharmaceutically acceptable addition salts and the stereochemically isomeric forms thereof, wherein
n is 0, 1 or 2; or
Z represents CH or CH₂;
—X— represents C₂₋₄alkynyl, C₂₋₄alkenyl, C₁₋₁₂alkyl optionally substituted with hydroxy or X represents a divalent radical of the formula 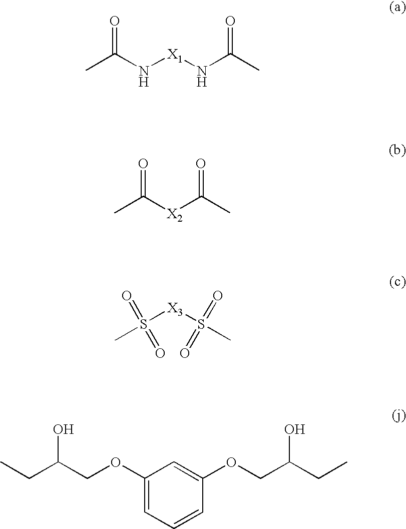
wherein; —X₁— represents C₁₋₁₂alkyl, phenyl or a divalent radical selected from the group consisting of 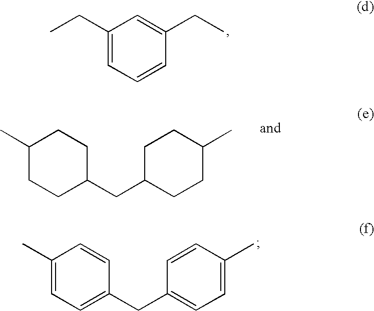
—X₂— represents C₁₋₁₂alkyl, C₁₋₄alkyloxyC₁₋₄alkyl, phenyl or a divalent radical of formula 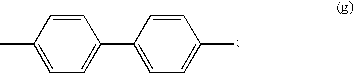
—X₃— represents phenyl or a divalent radical selected from the group consisting of 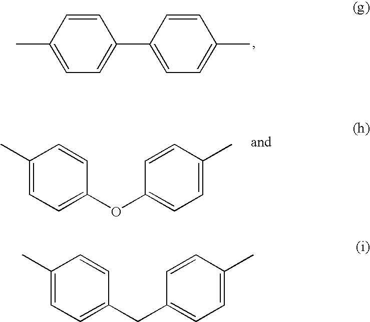
R¹ represents Ar¹, Ar²-carbonyl, Het², Ar³—C₁₋₄alkyloxy-, Ar⁴-oxy-, Het⁴-oxy-, or C₁₋₄alkyl substituted with one and where possible two or three substituents independently selected from NR³R⁴—, Het¹ or Ar⁶; or
R² represents hydroxy, benzyl, or C₁₋₄alkyloxy-;
R³ and R⁴ each independently represents hydrogen, C₁₋₄alkyl, C₁₋₄alkyloxy-, or Het³;
Het¹ represents a heterocycle selected from pyridinyl, pyrimidinyl, indolinyl, indolyl, benzimidazolyl, benzthiazolyl, benzisothiazolyl, benzisoxazolyl, thiazolyl, isothiazolyl or thiadiazolyl wherein said Het¹ is optionally substituted with one or where possible two or more substituents selected from the group consisting of hydroxy, halo, C₁₋₄alkyloxycarbonyl-, C₁₋₄alkyl-, C₁₋₄alkyloxy- and C₁₋₄alkyloxy-substituted with halo; in particular Het¹ represents a heterocycle selected from indolyl or pyridinyl;
Het² represents a heterocycle selected from indolyl, indolinyl, pyridinyl, pyrimidinyl, benzimidazolyl, benzoxazolyl, benzisoxazolyl, quinolinyl, quinazolinyl, quinoxalinyl, or oxodiazolyl wherein said Het² is optionally substituted with one or where possible two or more substituents selected from the group consisting of hydroxy, carbonyl, Ar⁵, halo, C₁₋₆alkyl- and C₁₋₄alkyloxy-;
Het³ represents a heterocycle selected from benzimidazolyl, benzoxazolyl, benzisoxazolyl, benzisothiazolyl or benzthiazolyl wherein said Het³ is optionally substituted with one or where possible two or more substituents selected from the group consisting of hydroxy, halo, C₁₋₆alkyl- and C₁₋₄alkyloxy-; in particular Het³ represents benzthiazolyl substituted with C₁₋₄alkyloxy-;
Het⁴ represents a heterocycle selected from pyrimidinyl, pyridinyl, indolinyl, indolyl, benzimidazolyl, benzoxazolyl, benzisoxazolyl, benzisothiazolyl or benzthiazolyl wherein said Het⁴ is optionally substituted with one or where possible two or more substituents selected from the group consisting of hydroxy, amino, mono or di-(C₁₋₄alkyl)amino, halo, C₁₋₆alkyl- and C₁₋₄alkyloxy-; in particular Het⁴ represents benzthiazolyl;
Ar¹ and Ar² each independently represent phenyl optionally substituted with halo, C₁₋₄alkyl-, C₁₋₄alkyloxy- or C₁₋₄alkyl substituted with one, two or three halo substituents; in particular Ar² or Ar¹ represents phenyl substituted with halo or trifluromethyl;
Ar³ and Ar⁴ each independently represent phenyl optionally substituted with halo, C₁₋₄alkyl-, C₁₋₄alkyloxy- or C₁₋₄alkyl substituted with one, two or three halo substituents; in particular Ar³ or Ar⁴ represents phenyl substituted with halo or trifluromethyl;
Ar⁵ represents phenyl optionally substituted with halo, C₁₋₆alkyl, C₁₋₄alkyloxy-, or C₃₋₆cycloalkyl-oxy-;
Ar⁶ represents phenyl optionally substituted with halo, C₁₋₆alkyl, C₁₋₄alkyloxy-, or C₃₋₆cycloalkyl-oxy-; provided however that;
for those compounds of formula (I) wherein —X— represents C₁₋₁₂alkyl optionally substituted with hydroxyl and R¹ represents Ar¹, for said compounds n represents 1 or 2; and
for those compounds of formula (I) wherein —X₂— represents phenyl, for said compounds R¹ represents Ar¹, Ar²-carbonyl, Ar³—C₁₋₄alkyloxy-, Ar⁴-oxy-, Het⁴-oxy-, or C₁₋₄alkyl substituted with one and where possible two or three substituents independently selected from NR³R⁴—, Het¹ or Ar⁶.

2. A compound according to claim 1 wherein;
n is 0, 1 or 2;
Z represents —CH— or —CH₂—;
—X— represents C₂₋₄alkynyl, C₁₋₁₂alkyl optionally substituted with hydroxy or X represents a divalent radical of the formula 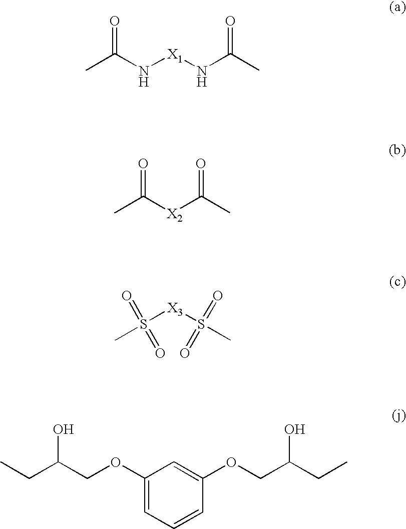
wherein; —X₁— represents C₁₋₁₂alkyl, phenyl or a divalent radical selected from the group consisting of 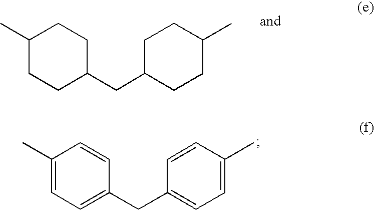
—X₂— represents C₁₋₁₂alkyl, phenyl or a divalent radical of formula 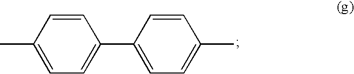
—X₃— represents phenyl or a divalent radical selected from the group consisting of 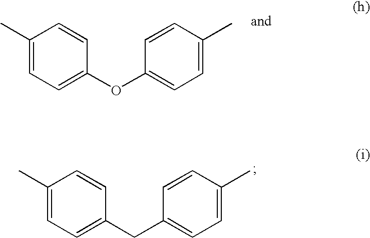
R¹ represents Ar¹, Ar²-carbonyl, Het², Ar³—C₁₋₄alkyloxy-, Het⁴-oxy- or C₁₋₄alkyl substituted with one or where possible two or three substituents independently selected from NR³R⁴ or Het¹;
R² represents hydroxyl;
R³ and R⁴ each independently represent hydrogen or Het³;
Het¹ represents a heterocycle selected from indolinyl, indolyl, pyridinyl, benzthiazolyl or benzisothiazolyl wherein said Het¹ is optionally substituted with one or where possible two or more substituents selected from halo, hydroxyl or C₁₋₄alkyloxy;
Het² represents a heterocycle selected from indolyl, indolinyl, benzoxazolyl, benzisoxazolyl or oxodiazolyl wherein said Het² is optionally substituted with one or where possible two or more substituents selected from halo, hydroxyl, Ar⁵ or C₁₋₆alkyl;
Het³ represents a heterocycle selected from benzthiazolyl or benzisothiazolyl, wherein said Het³ is optionally substituted with one or where possible two or more substituents selected from halo, hydroxyl or C₁₋₄alkyloxy;
Het⁴ represents a heterocycle selected from benzthiazolyl or benzisothiazolyl, wherein said Het³ is optionally substituted with one or where possible two or more substituents selected from halo, hydroxyl or C₁₋₄alkyloxy;
Ar¹ and Ar² each independently represent phenyl optionally substituted with one, two or more substituents selected from halo or C₁₋₄alkyl substituted with one, two or three halo substituents;
Ar³ and Ar⁴ each independently represent phenyl optionally substituted with one, two or more substituents selected from halo or C₁₋₄alkyl substituted with one, two or three halo substituents; and
Ar⁵ represents phenyl optionally substituted with C₁₋₄alkyloxy-, or C₃₋₆cycloalkyl-oxy-; provided however that;
for those compounds of formula (I) wherein —X— represents C₁₋₁₂alkyl optionally substituted with hydroxyl and R¹ represents Ar¹, for said compounds n represents 1 or 2; and
for those compounds of formula (I) wherein —X₂— represents phenyl, for said compounds R¹ represents Ar¹, Ar²-carbonyl, Ar³—C₁₋₄alkyloxy-, Ar⁴-oxy-,
Het⁴-oxy-, or C₁₋₄alkyl substituted with one and where possible two or three substituents independently selected from NR³R⁴—, Het¹ or Ar⁶.

3. A compound according to claims 1 wherein;
n is 0, 1 or 2; Z represents CH or CH₂;
—X— represents C₂₋₄alkynyl, C₁₋₁₂alkyl optionally substituted with hydroxy or X represents a divalent radical of the formula 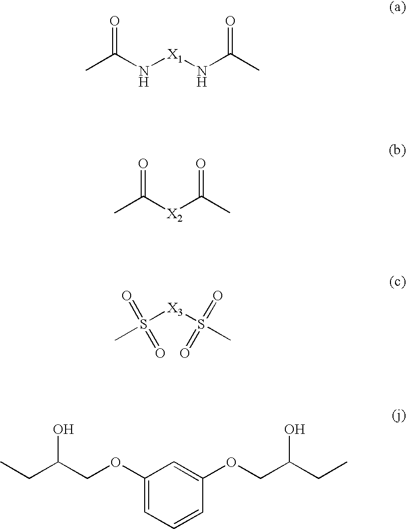
wherein; —X₁— represents C₁₋₁₂alkyl, phenyl or a divalent radical selected from the group consisting of 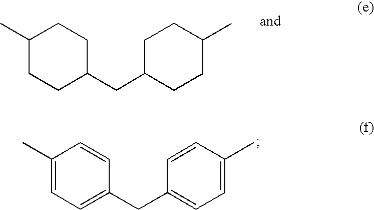
—X₂— represents C₁₋₁₂alkyl, phenyl or a divalent radical of formula 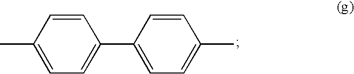
—X₃— represents phenyl or a divalent radical selected from the group consisting of 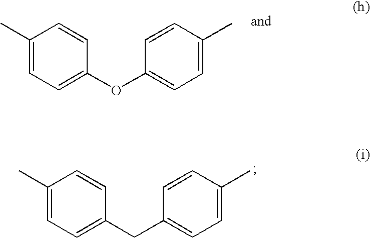
R¹ represents Ar¹, Ar²-carbonyl, Het², Ar³—C₁₋₄alkyloxy-, Het⁴-oxy- or C₁₋₄alkyl substituted with one or where possible two or three substituents independently selected from NR³R⁴ or Het¹;
R² represents hydroxyl;
R³ and R⁴ each independently represent hydrogen or Het³;
Het¹ represents a heterocycle selected from indolyl or benzthiazolyl;
Het² represents a heterocycle selected from indolyl, pyridinyl, benzisoxazolyl or oxodiazolyl wherein said Het² is optionally substituted with one or where possible two or more substituents selected from halo, Ar⁵ or C₁₋₆alkyl;
Het³ represents benzthiazolyl wherein said Het³ is optionally substituted with one or where possible two or more substituents selected from halo or C₁₋₄alkyloxy; in particular Het³ represents benzthiazolyl substituted with one or more C₁₋₄alkyloxy substituents;
Het⁴ represents benzthiazolyl;
Ar¹ and Ar² each independently represent phenyl optionally substituted with one, two or more substituents selected from halo or C₁₋₄alkyl substituted with one, two or three halo substituents;
Ar³ and Ar⁴ each independently represent phenyl optionally substituted with one, two or more C₁₋₄alkyl substituents, said C₁₋₄alkyl substituted with one, two or three halo substituents; and
Ar⁵ represents phenyl optionally substituted with C₁₋₄alkyloxy-, or C₃₋₆cycloalkyl-oxy-; provided however that;
for those compounds of formula (I) wherein —X— represents C₁₋₁₂alkyl optionally substituted with hydroxyl and R¹ represents Ar¹, for said compounds n represents 1 or 2; and
for those compounds of formula (I) wherein —X₂— represents phenyl, for said compounds R¹ represents Ar¹, Ar²-carbonyl, Ar³—C₁₋₄alkyloxy-, Ar⁴-oxy-, Het⁴-oxy-, or C₁₋₄alkyl substituted with one and where possible two or three substituents independently selected from NR³R⁴—, Het¹ or Ar⁶.

4. A compound according to claims 1 wherein;
n is 1;
—X— represents C₁₋₁₂alkyl optionally substituted with hydroxyl or —X— represents a divalent radical of the formula 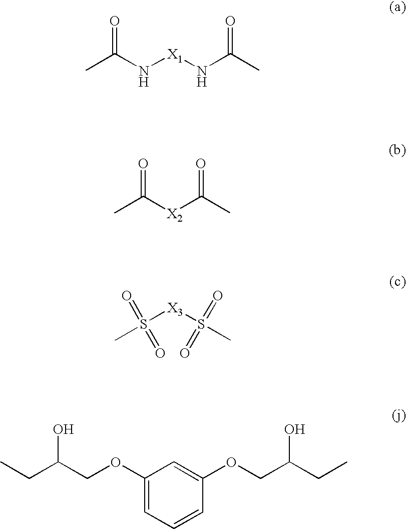
wherein; —X₁— represents C₁₋₁₂alkyl, phenyl or the divalent radical 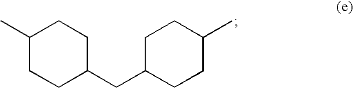
—X₂— represents C₁₋₁₂alkyl;
—X³— represents 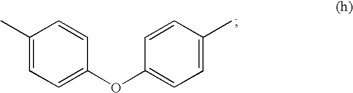
R¹ represents Ar¹;
R² represents hydroxyl;
Ar¹ represents phenyl substituted with two or more substituents selected from halo or C₁₋₄alkyl substituted with one, two or three halo substituents.

5. A compound according to claims 1 wherein;
Het¹ represents a heterocycle selected from indolyl or pyridinyl;
Het³ represents benzthiazolyl substituted with C₁₋₄alkyloxy-;
Het⁴ represents benzthiazolyl;
Ar² represents phenyl substituted with halo or trifluromethyl.

6. A compound as claimed in claim 1 selected from those of formulae (A), (B), (C), (D), (E), (F), (G), (H) and (I) below:

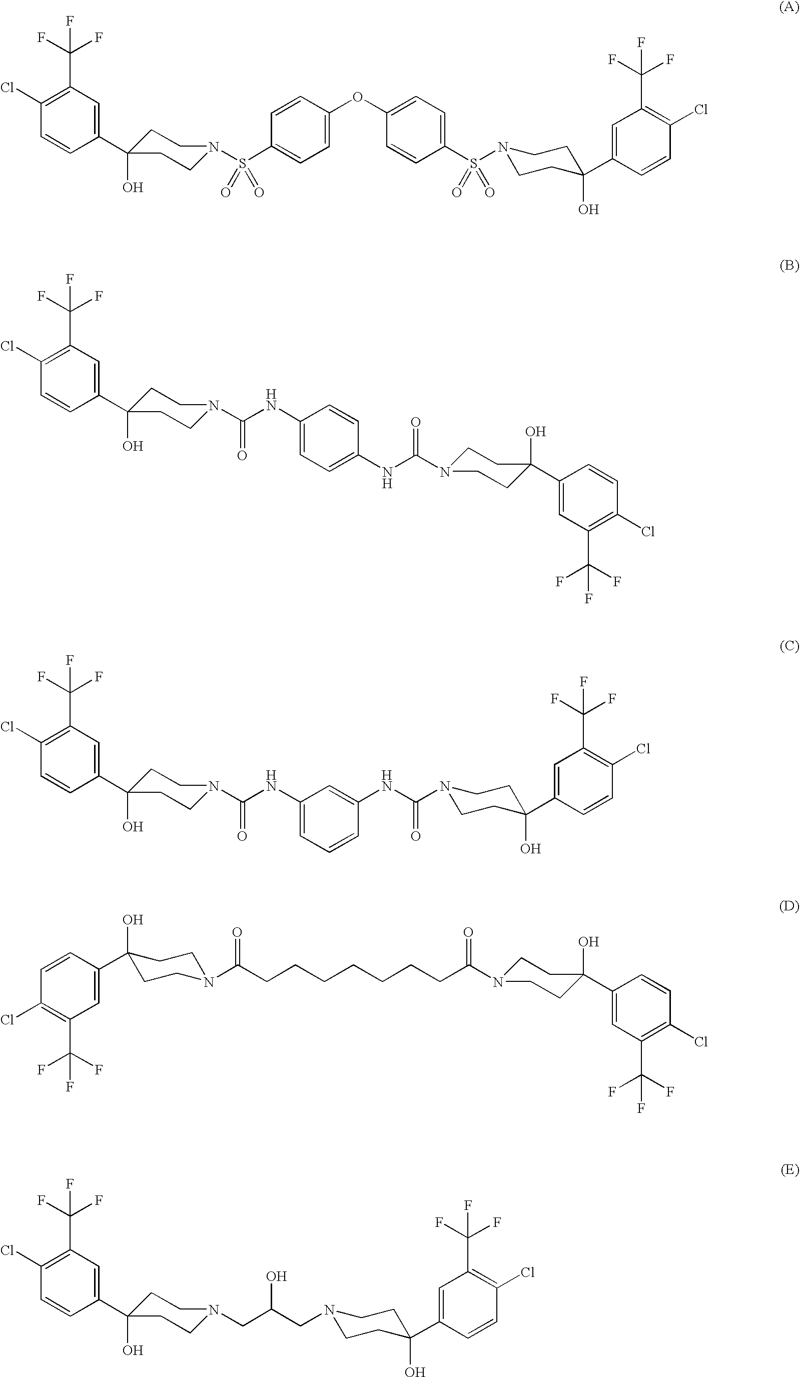

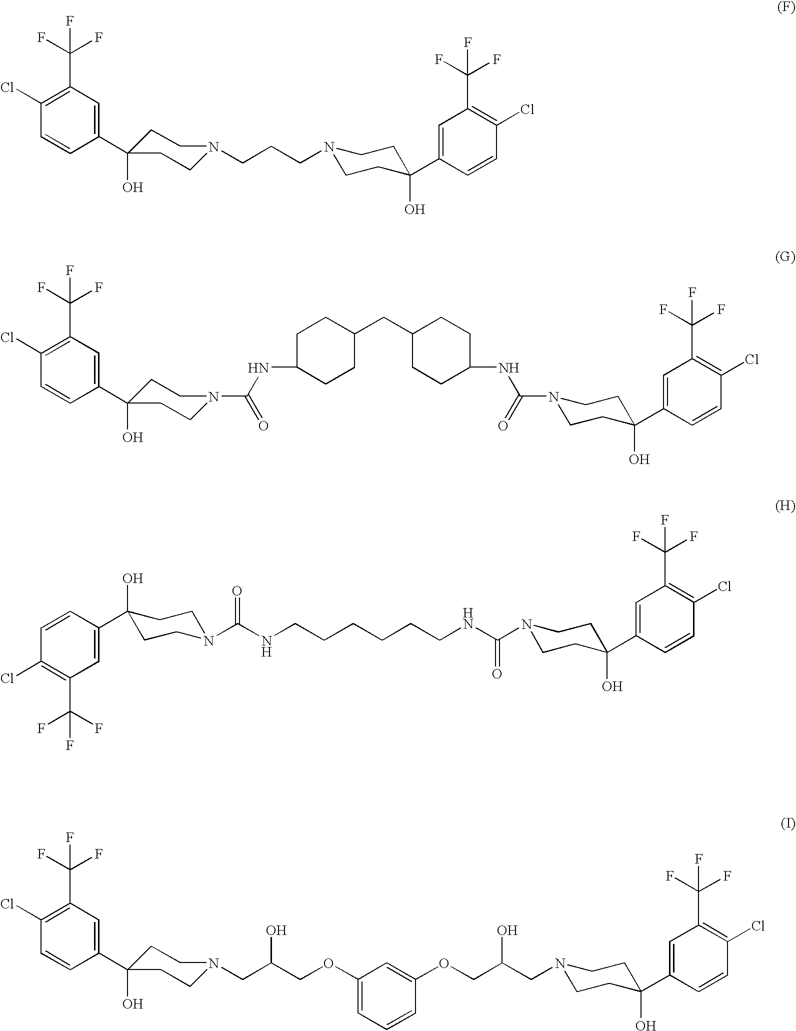

7. A pharmaceutical composition comprising a pharmaceutically acceptable carrier and, as active ingredient, a therapeutic effective amount of a compound as described in claims 1.

8. (canceled)

9. (canceled)

10. The method of claim 13, wherein the pain is post-operative pain.

11. A method of treating or preventing neurodegenerative mediated disorders comprising administering to a host in need thereof or effective amount of a compound of claim 1.

12. The method according to claim 11 wherein the neurodegenerative mediated-disorder is selected from stroke, Alzheimer's disease, Parkinson's disease, Huntington's disease, amyotrophic lateral sclerosis, Pick's disease, fronto-temporal dementia, progressive nuclear palsy, corticobasal degeneration, cerebro-vascular dementia, multiple system atrophy, argyrophilic grain dementia, other tauopathies, age-related macular degeneration, narcolepsy, motor neuron diseases, prion diseases, traumatic nerve injury and repair, and multiple sclerosis.

13. A method for treating pain comprising administering to a host in need thereof an effective amount of a compound as claimed in claim 1.

14. A method of treating pathologies associated with neuronal death, stroke, Alzheimer's disease, Parkinson's disease, Huntington's disease, amyotrophic lateral sclerosis, Pick's disease, fronto-temporal dementia, progressive nuclear palsy, corticobasal degeneration, cerebro-vascular dementia, multiple system atrophy, argyrophilic grain dementia, other tauopathies, and further conditions involving neurodegenerative processes are for instance, age-related macular degeneration, narcolepsy, motor neuron diseases, prion diseases, traumatic nerve injury and repair, and multiple sclerosis comprising administering to a host in need thereof an effective amount of a compound of claim 1.
